(19) United States
(12) Patent Application Publication
(10) Pub. No.: US 2018/0085247 A1
(43) Pub. Date: Mar. 29, 2018

(54) Systems, Methods, and Devices for Improving Airflow-Related Sleep Problems

(71) Applicant: SLEEP BETTER PRODUCTS, INC., Tampa, FL (US)

(72) Inventors: JOSEPH JUDE TRAINOR, TAMPA, FL (US); JOHN JOSEPH TRAINOR, DURHAM, NC (US); MICHAEL ROSS DICKSON, DURHAM, NC (US)

(21) Appl. No.: 15/716,599

(22) Filed: Sep. 27, 2017

Related U.S. Application Data
(60) Provisional application No. 62/401,209, filed on Sep. 29, 2016.

(51) Int. Cl. A61F 5/56; A62B 9/06

(52) U.S. Cl. CPC A61F 5/566; A61F 2005/563; A62B 9/06

(57) ABSTRACT

Various embodiments of sleep-aid mouthpieces and related accessories and methods are provided. One embodiment of a sleep-aid mouthpiece comprises: a U-shaped, in-mouth member configured to be worn in a user's mouth, the U-shaped, in-mouth member comprising a middle portion from which a pair of leg portions extend inwardly to respective end portions inside the user's mouth; a front member extending from an outer surface of the U-shaped, in-mouth member and disposed outside of the user's mouth; a pair of lateral breathing ports extending from outside the user's mouth through the front member and the U-shaped, in-mouth member and into the user's mouth; and an anterior tongue advancement device disposed on an inner surface of the middle portion of the U-shaped, in-mouth member, the anterior tongue advancement device comprising one or more flexible members for receiving and holding the user's tongue in a forward position.

### CROSS-REFERENCE TO RELATED APPLICATION

This application claims the benefit of the priority of U.S. Provisional Patent Application Ser. No. 62/401,209 entitled “Systems, Methods, and Devices for Improving Airflow-Related Sleep Problems” and filed on Sep. 29, 2016 (Attorney Ref. No. 19154.0001U1), which is hereby incorporated by reference in its entirety.

### DESCRIPTION OF THE RELATED ART

Sleeping is a basic human need. Sleep plays a vital role in good health and wellbeing throughout your life. Getting sufficient quality sleep can help protect mental and physical health and promote a better quality of life. Individuals who experience sleep insufficiency are more likely to suffer from chronic diseases such as hypertension, diabetes, depression, and obesity, as well as from cancer, increased mortality, and reduced quality of life and productivity. Sleep insufficiency may be caused by any number of factors (e.g., environmental, physical characteristics, comfort, psychological, health, medical, etc.), although a significant or aggravating cause for a large number of individuals is generally due to insufficient airflow that may promote snoring, affect breathing (e.g., volume, frequency, patterns, etc.), or otherwise negatively impact the quality and/or frequency of sleep. Airflow-related sleep insufficiency may range in frequency and degree on a case-by-case basis, and may change over time depending on individual and external factors. For example, some individuals may suffer from a sleep disorder, such as, obstructive sleep apnea, while others may not.

Many solutions have been proposed to address this widespread yet varying problem. For the treatment of obstructive sleep apnea, the most common treatment involves a continuous positive airway pressure (CPAP) machine. CPAP machines comprise a mask that fits over the nose and/or mouth with straps to keep the mask in place. A tube connects the mask to an external motor that blows air into the tube, providing mild pressure that can prevent the airway from collapsing or becoming blocked. CPAP machines have several disadvantages. CPAP machines are expensive and cumbersome. Common problems include a leaky mask, difficulty in falling asleep due to machine noise and/or mask discomfort, and a dry mouth or nose.

For those who cannot afford a CPAP machine or otherwise reject this treatment due to discomfort, ineffectiveness, or other problems, there are also various intra-oral and dental appliances that are commonly used to address airflow-related sleep insufficiency. Such devices are worn inside of the mouth and designed to increase airflow by repositioning of the jaw, moving the mandible, lifting the soft palate, or moving the tongue forward. Intra-oral and dental appliances, however, have many disadvantages. They typically require initial customization and ongoing maintenance by a trained dental professional to properly align the in-mouth structures to provide the proper displacement of the jaw, mandible, or soft palate. Like CPAP treatment, these devices can be relatively expensive, and many individuals have difficulty falling asleep and complain of pain, discomfort, and/or difficulty breathing due to the in-mouth structures. Structures used to displace the jaw, mandible, or soft palate may not initially cause discomfort but can cause fatigue or pain over time.

There are known oral devices that use suction to advance the tongue forward to improve airflow. U.S. Pat. No. 6,494,209 discloses a mouthpiece comprising an integrally-formed tongue-receiving cavity that is positioned to snugly receive the user's tongue in a forward position. The tongue-receiving cavity includes a small suction channel that is connected to an external suction source using tubing. Suction-based tongue advancement, such as disclosed in U.S. Pat. No. 6,494,209, has many disadvantages. For example, to properly insert the tongue into the tongue-receiving cavity, the user may be required to use a hand and may have difficulty in obtaining a comfortable position. Due to the suction, it may also be cumbersome to easily remove the tongue from the tongue-receiving cavity without the user's hands. Furthermore, suction applied at the tip of the tongue may limit blood flow, create some discomfort or numbness, and may even temporarily reduce taste sensitivity after use.

Accordingly, despite these widespread problems and the availability of numerous commercial products that propose to address it, there remains a need in the art for improved systems, methods, and devices for addressing airflow-related sleep insufficiency, snoring, etc.

### SUMMARY OF THE DISCLOSURE

Various embodiments of sleep-aid mouthpieces and related accessories and methods are provided. One embodiment of a sleep-aid mouthpiece comprises: a U-shaped, in-mouth member configured to be worn in a user's mouth, the U-shaped, in-mouth member comprising a middle portion from which a pair of leg portions extend inwardly to respective end portions inside the user's mouth; a front member extending from an outer surface of the U-shaped, in-mouth member and disposed outside of the user's mouth; a pair of lateral breathing ports extending from outside the user's mouth through the front member and the U-shaped, in-mouth member and into the user's mouth; and an anterior tongue advancement device disposed on an inner surface of the middle portion of the U-shaped, in-mouth member, the anterior tongue advancement device comprising one or more flexible members for receiving and holding the user's tongue in a forward position.

Another embodiment is a portable airflow accessory for a sleep-aid mouthpiece. The portable airflow accessory comprises a housing containing a source of airflow powered by a battery and an adapter. The adapter is disposed on the housing for removably securing to a sleep-aid mouthpiece. The adapter comprises a pair of lateral members configured to be removably secured in a pair of corresponding lateral breathing ports disposed on the sleep-aid mouthpiece. The adapter further comprises a center securing member configured to be removably secured in a central passageway disposed on the sleep-aid mouthpiece.

### BRIEF DESCRIPTION OF THE DRAWINGS

In the Figures, like reference numerals refer to like parts throughout the various views unless otherwise indicated. For reference numerals with letter character designations such as “102A” or “102B”, the letter character designations may differentiate two like parts or elements present in the same Figure. Letter character designations for reference numerals may be omitted when it is intended that a reference numeral to encompass all parts having the same reference numeral in all Figures.

FIG. 1 is a perspective view of an embodiment of a sleep-aid mouthpiece.

FIG. 2 is a front view of the sleep-aid mouthpiece in FIG. 1.

FIG. 3 is a side view of the sleep-aid mouthpiece of FIG. 1.

FIG. 4 illustrates a cross-sectional view through the central passageway of the sleep-aid mouthpiece of FIGS. 1-3.

FIG. 5 is a top view of the sleep-aid mouthpiece of FIGS. 1-3.

FIG. 6 is a perspective view of an embodiment of an anterior tongue advancement device that may be removably inserted into the central passageway of the sleep-aid mouthpiece of FIGS. 1-3.

FIG. 7 illustrates the anterior tongue advancement device of FIG. 6 inserted into an inner opening of the central passageway of the sleep-aid mouthpiece of FIGS. 1-3.

FIG. 8 is a perspective view of an embodiment of an external airflow device that may be removably inserted into an outer opening of the central passageway of the sleep-aid mouthpiece of FIGS. 1-3.

FIG. 9 illustrates the external airflow device removably secured to the sleep-aid mouthpiece of FIGS. 1-3.

FIG. 10a is a schematic view of an embodiment of a mouthpiece adapter disposed on the external airflow device.

FIG. 10b illustrates the mouthpiece adapter of FIG. 10a removably secured to the sleep-aid mouthpiece of FIGS. 1-3.

FIG. 11a illustrates a cross-sectional view of the mouthpiece adapter of FIG. 10a and the sleep-aid mouthpiece of FIGS. 1-3.

FIG. 11b illustrates a cross-sectional view of the mouthpiece adapter of FIG. 10a removably secured to the sleep-aid mouthpiece of FIGS. 1-3.

FIG. 12 illustrates a cross-sectional top view of an embodiment of a sleep-aid mouthpiece and a mouthpiece adapter disposed on the external airflow device.

FIG. 13 illustrates the mouthpiece adapter of FIG. 12 removably secured to the sleep-aid mouthpiece.

FIG. 14 illustrates an exemplary embodiment of a nasal spreader removably secured to a nasal spreader latch disposed on the sleep-aid mouthpiece.

FIG. 15 illustrates an embodiment of a nasal spreader that may be incorporated with the sleep-aid mouthpiece of FIGS. 1-3.

FIG. 16a illustrates another embodiment of a nasal spreader that may be incorporated with the sleep-aid mouthpiece of FIGS. 1-3.

FIG. 16b illustrates the nasal spreader of FIG. 16a in a flexed position to be inserted in the user's nostrils.

FIG. 17a illustrates another embodiment of a nasal spreader that may be incorporated with the external airflow device.

FIG. 17b is a cross-sectional view of the nasal spreader of FIG. 17a illustrating the inner longitudinal passageway through the ends of the nasal spreader.

FIG. 18a illustrates another embodiment of an external airflow device comprising an airflow port for connecting to the hollow nasal spreader of FIGS. 17a and 17b.

FIG. 18b illustrates the airflow port on the external airflow device mated with the airflow port on the hollow nasal spreader.

FIG. 19 is a perspective view of another embodiment of a sleep-aid mouthpiece that may be used in the multi-modal sleep-aid system of FIGS. 1-18 and incorporated with an anti-drool device.

FIG. 20 is a front view of the sleep-aid mouthpiece of FIG. 19.

FIG. 21 is a top view of the sleep-aid mouthpiece of FIG. 19.

FIG. 22 is a rear view of another embodiment of a sleep-aid mouthpiece operatively coupled with an exemplary anti-drool device.

FIG. 23 is a bottom view of the sleep-aid mouthpiece of FIG. 22.

FIG. 24 is a side view of the sleep-aid mouthpiece of FIG. 22 operatively coupled with the exemplary anti-drool device.

FIG. 25 is a perspective view of an embodiment of an anti-drool device.

FIG. 26 is a side view of the anti-drool device of FIG. 25.

FIG. 27 is a rear view of the sleep-aid mouthpiece of FIG. 22 illustrating an opening in the anterior tongue advancement device for installing the anti-drool device.

FIG. 28 is a front view of the sleep-aid mouthpiece of FIG. 19 illustrating an alignment/retaining feature on a lower portion of the inner wall of the central passageway for receiving and securing the anti-drool device.

FIG. 29 is a front view of the sleep-aid mouthpiece of FIG. 19 illustrating the anti-drool device disposed in a secured position.

FIG. 30 illustrates another embodiment of an external airflow device removably secured to a sleep-aid mouthpiece.

FIG. 31 illustrates an embodiment of a removable air filter cover disposed on the housing of the external airflow device of FIG. 30.

FIG. 32 is a back view of the removable air filter cover of FIG. 31.

FIG. 33 illustrates an embodiment of a baffled nostril cone accessory that may be integrated with the external airflow device of FIG. 30.

FIG. 34 is a side view of the baffled nostril cone accessory of FIG. 33.

FIG. 35 is a top view of the baffled nostril cone accessory of FIG. 33.

FIG. 36 is a bottom view of the baffled nostril cone accessory of FIG. 33.

FIG. 37 is a perspective view of another embodiment of a mouthpiece adapter disposed on the external airflow device of FIG. 30.

FIG. 38 is a front view of the mouthpiece adapter of FIG. 37.

FIG. 39 is a perspective view of another embodiment of a tethered nasal spreader that may be removably attached to the sleep-aid mouthpiece of FIGS. 19 & 22.

FIG. 40 is a side view of the tethered nasal spreader of FIG. 39.

### DETAILED DESCRIPTION

The word “exemplary” is used herein to mean “serving as an example, instance, or illustration.” Any aspect described herein as “exemplary” is not necessarily to be construed as preferred or advantageous over other aspects.

Various embodiments of sleep-aid systems, methods, and devices are disclosed for improving airflow-related sleep problems. As mentioned above, air-flow-related sleep insufficiency may be caused by any number of factors (e.g., environmental, physical characteristics, comfort, psychological, health, medical, etc.). A significant or aggravating cause for a large number of individuals is generally due to insufficient airflow that may promote snoring, affect breathing (e.g., volume, frequency, patterns, etc.), or otherwise negatively impact the quality and/or frequency of sleep. Airflow-related sleep insufficiency may range in frequency and degree on a case-by-case basis, and may change over time depending on individual and external factors.

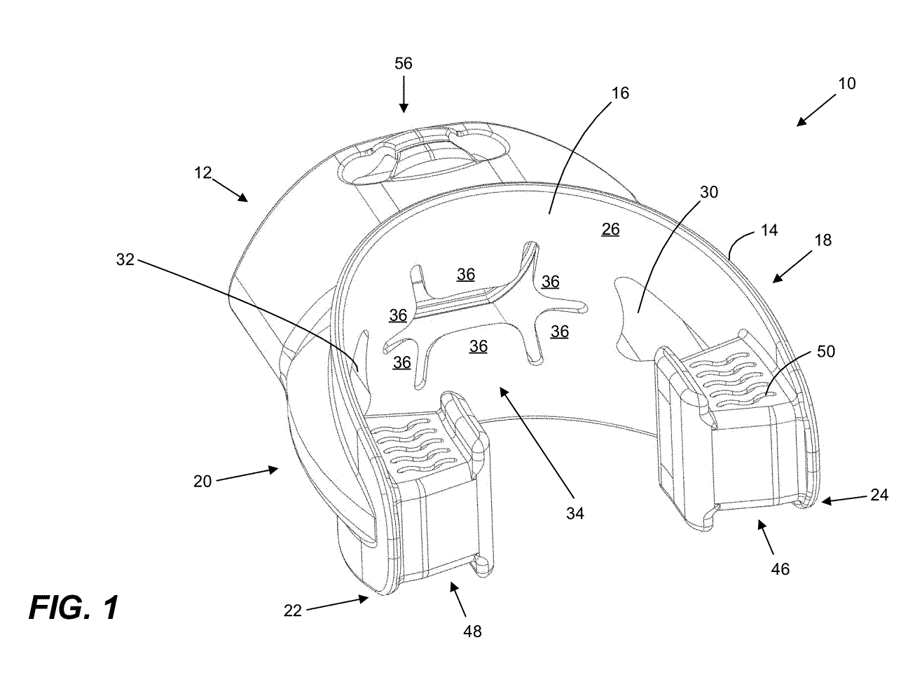

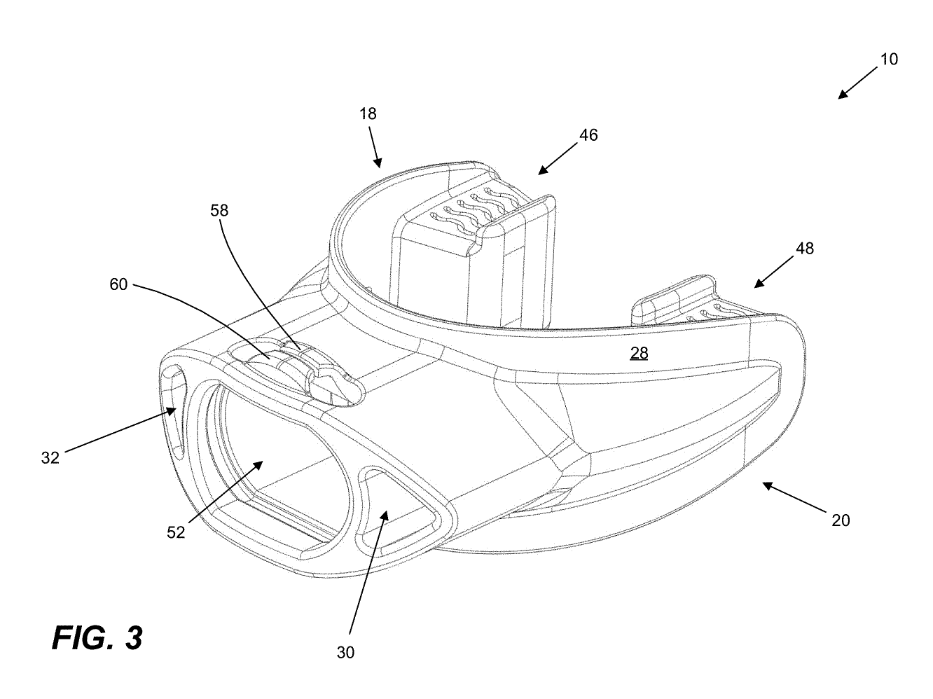

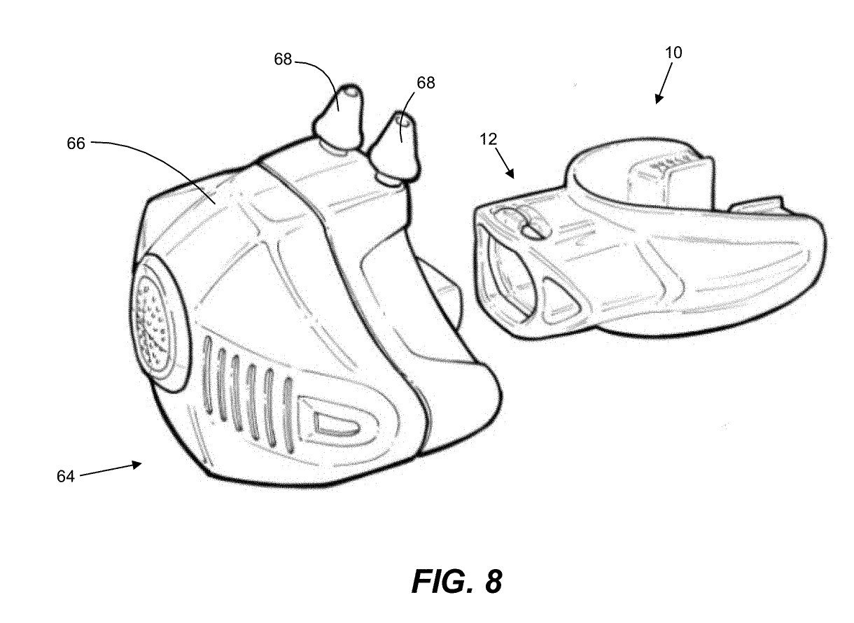

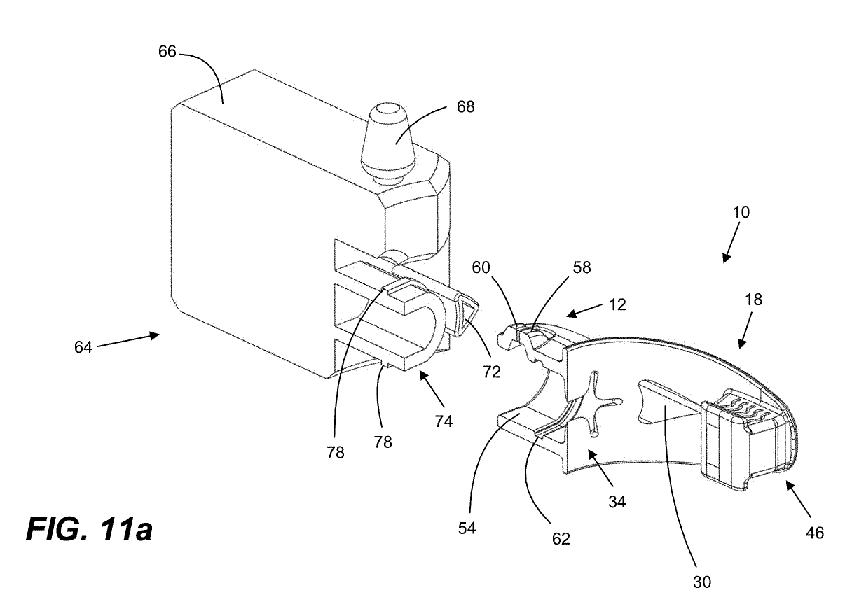

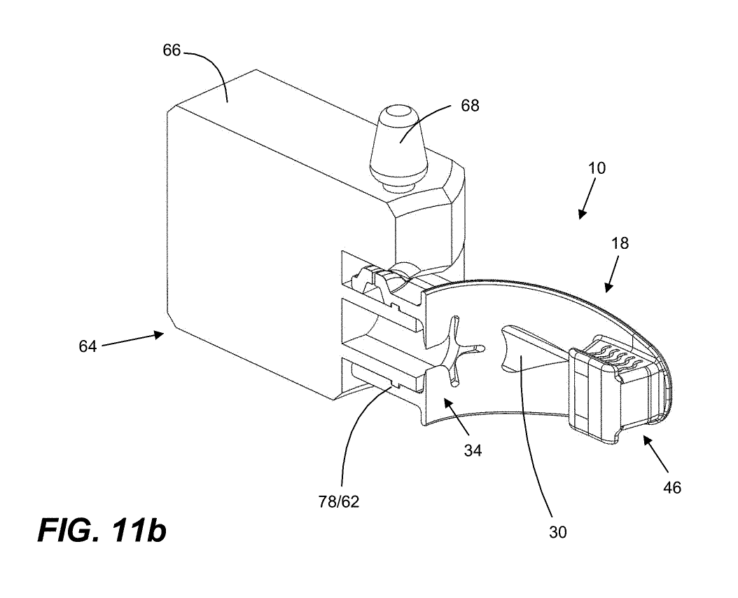

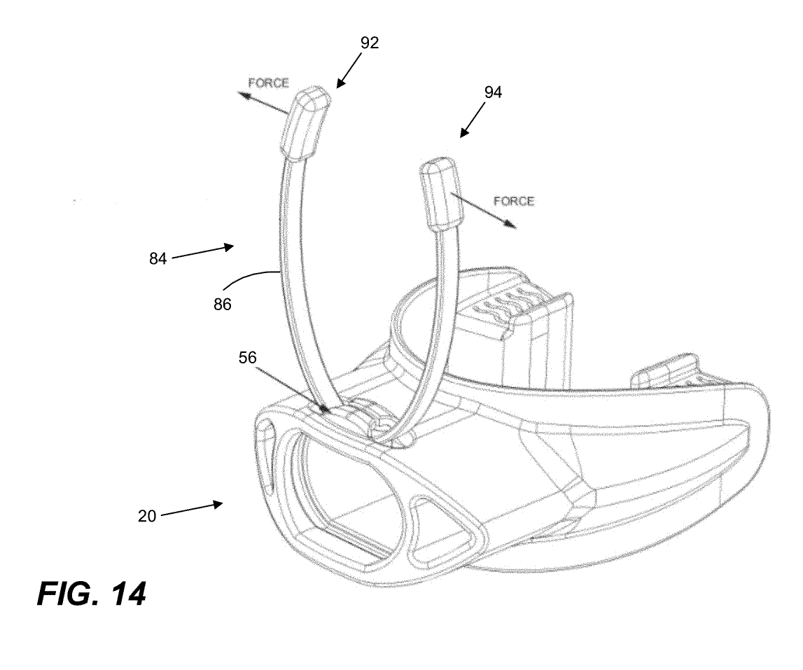

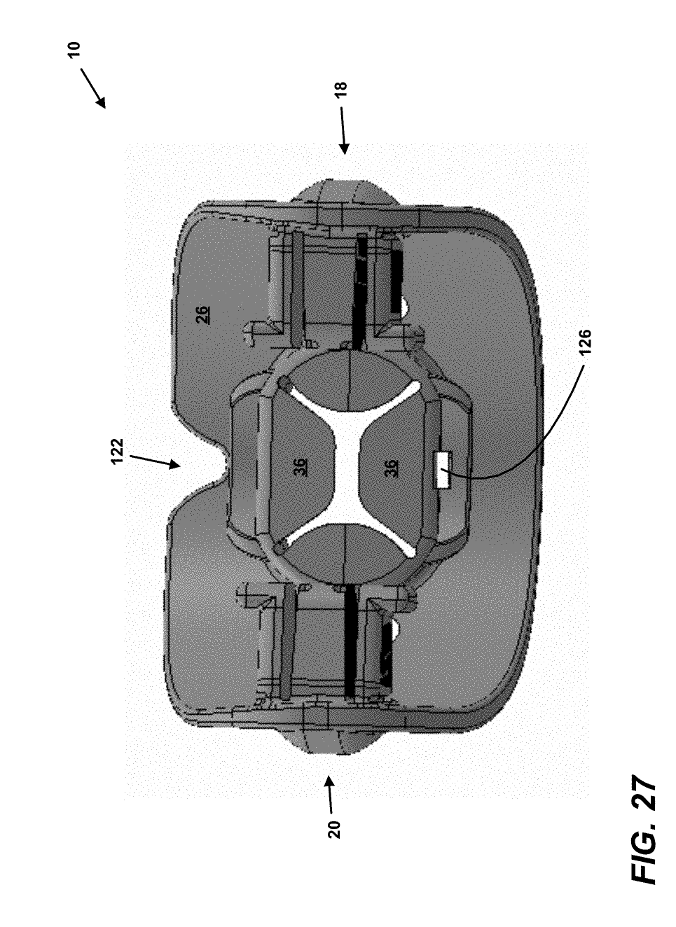

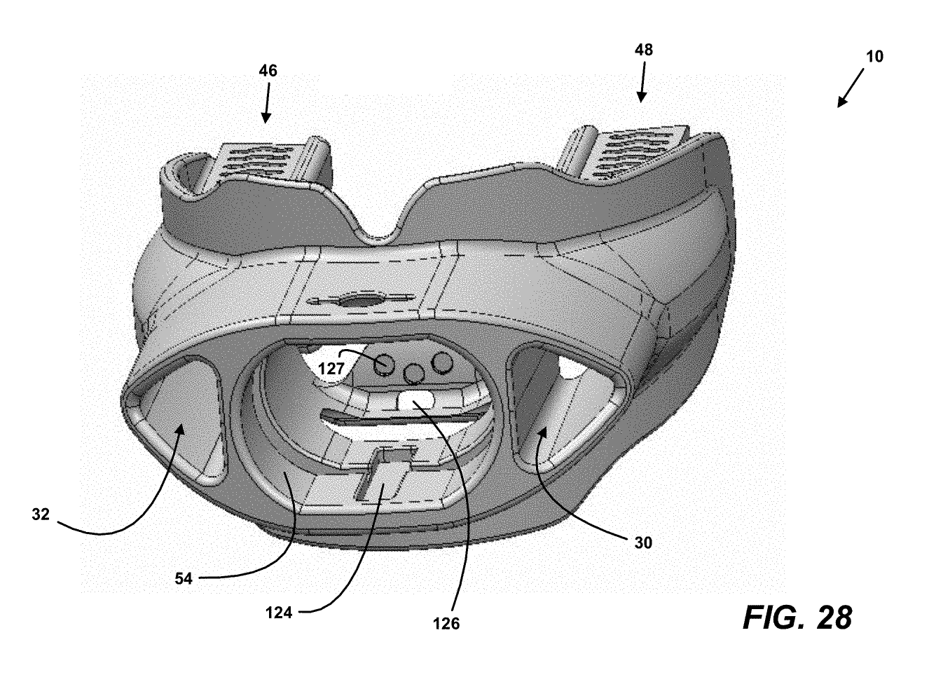

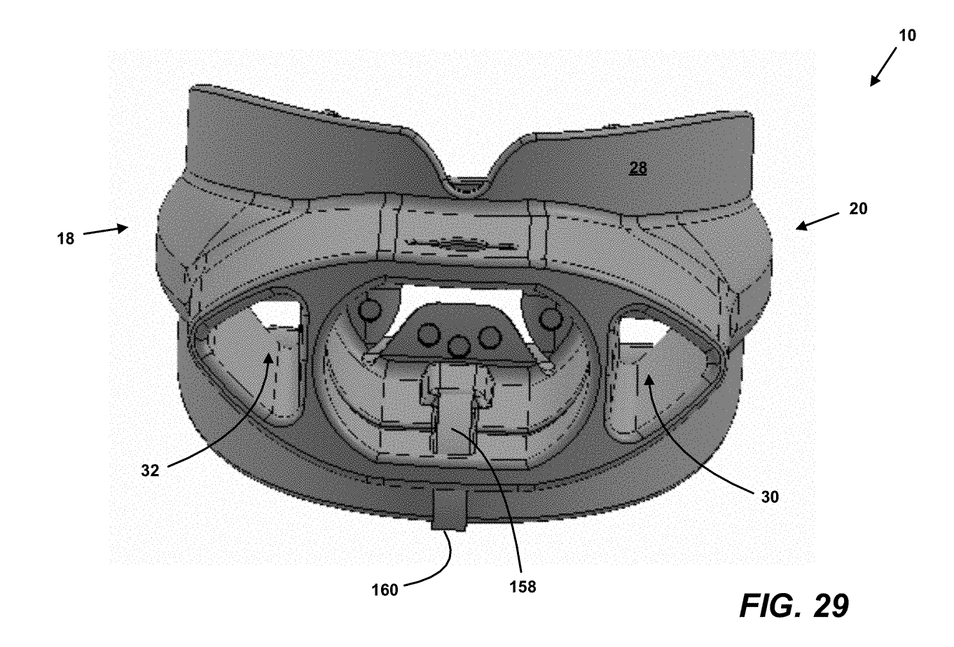

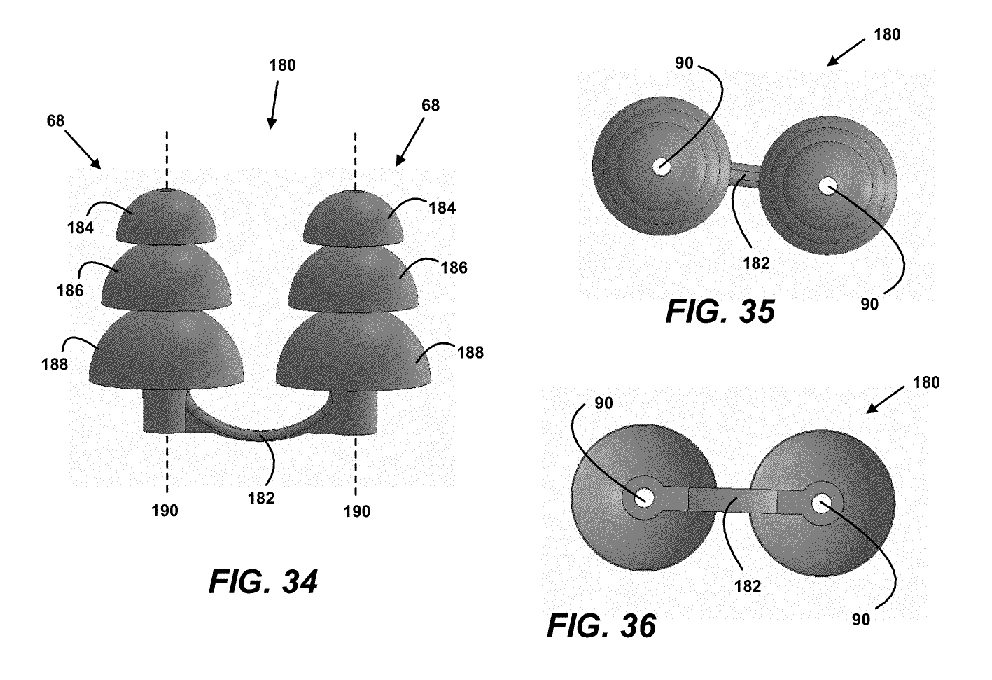

In this regard, it should be appreciated that the sleep-aid systems, methods, and devices described below in connection with FIGS. 1-42 may be used to address various airflow-flow sleep problems, including, for example, snoring of any degree and/or frequency regardless the causes and/or factors, as well as sleeping disorders, such as, obstructive sleep apnea. By way of introduction, in an exemplary embodiment, a sleep-aid mouthpiece 10 (FIGS. 1-7), an external airflow device 64 (FIGS. 8-13), and a nasal spreader 84 (FIGS. 14-18) comprise components of an integrated multi-modal sleep-aid system. The multi-modal sleep-aid system enables someone who is suffering from airflow-related sleep problems to selectively use the various components, devices, accessories, etc. described herein (e.g., the sleep-aid mouthpiece 10, the external airflow device 64, the nasal spreader 84, the anti-drool device 150, etc.) in various combinations or modalities. The various embodiments of each system component are described below in more detail. However, as an introductory matter, the various modalities provided by the multi-modal sleep-aid system will be described.

The multi-modal sleep-aid system promotes a better night sleep by opening the airway in the back of the throat, advancing the tongue to further open the airway, spreading the nostrils for better nasal airflow, and introducing airflow into the mouth or nose. The four components of the multi-modal system comprise: (1) a sleep-aid mouthpiece 10; (2) a nasal spreader 84; (3) a tongue advancement device; and (4) the external airflow device 64. The sleep-aid mouthpiece 10 is a foundational component of the multi-modal sleep-aid system and, therefore, may be used in each of the modalities. As described below in more detail, the sleep-aid mouthpiece 10 keeps the mouth open while preventing teeth grinding and provides the various means for integrating and/or attaching the nasal spreader 84, the external airflow device 64, and the tongue advancement device.

A first modality comprises the sleep-aid mouthpiece 10 used as a standalone device. The tongue advancement device may be integrated with the sleep-aid mouthpiece 10 (e.g., integral anterior tongue advancement device 34). The first modality may be useful in various situations, such as, for example, to prevent bruxism, where nasal passages become restricted or blocks, or for use by a relatively mild or occasional snorer. The height of the bite blocks 46 and 48 may gently separate the upper and lower jaws to open the airway.

A second modality comprises the sleep-aid mouthpiece 10 with an integrated tongue advancement device or a removable anterior tongue advancement accessory 38. A user may choose to operate with or without the tongue advancement device. It should be appreciated that the tongue advancement device may be easily secured with the sleep-aid mouthpiece 10. The second modality may be particularly useful for relatively medium to heavy snorers that have airway obstruction due to relaxation of the tongue. The tongue is gently retained in the anterior tongue advancement device in a forward position to open the back of the throat airway.

A third modality comprises the sleep-aid mouthpiece 10 and the nasal spreader 84. The nasal spreader 84 may be secured to the sleep-aid mouthpiece 10 via, for example, an attachment mechanism 56 disposed on an upper surface of a front member 12. This modality may be particularly useful where nasal passages are restricted or obstructed by enhancing airflow through the nose by widening the nostrils. It should be appreciated that the nasal spreader 84 may be used to open nostril airways either by attachment to the sleep-aid mouthpiece 10 or as an independent device.

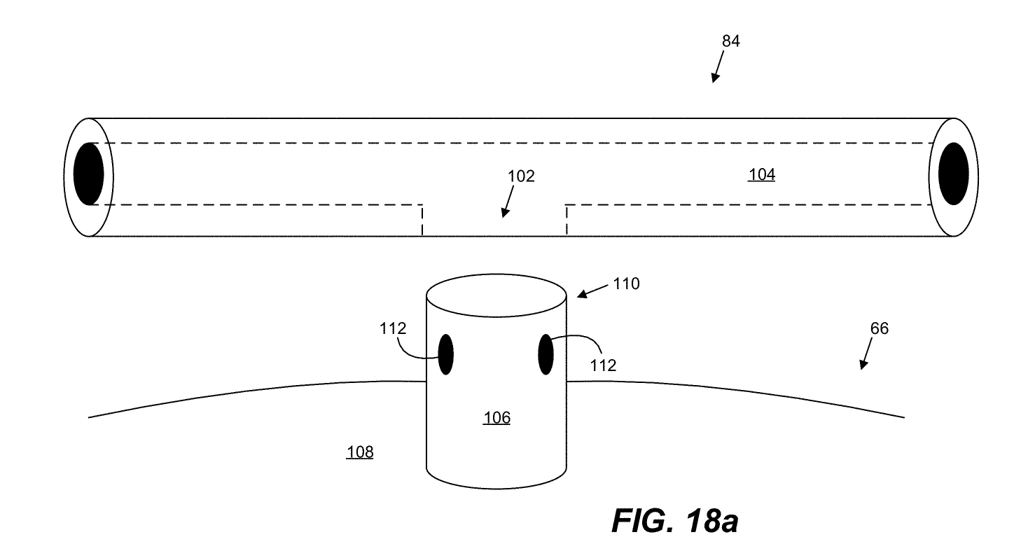

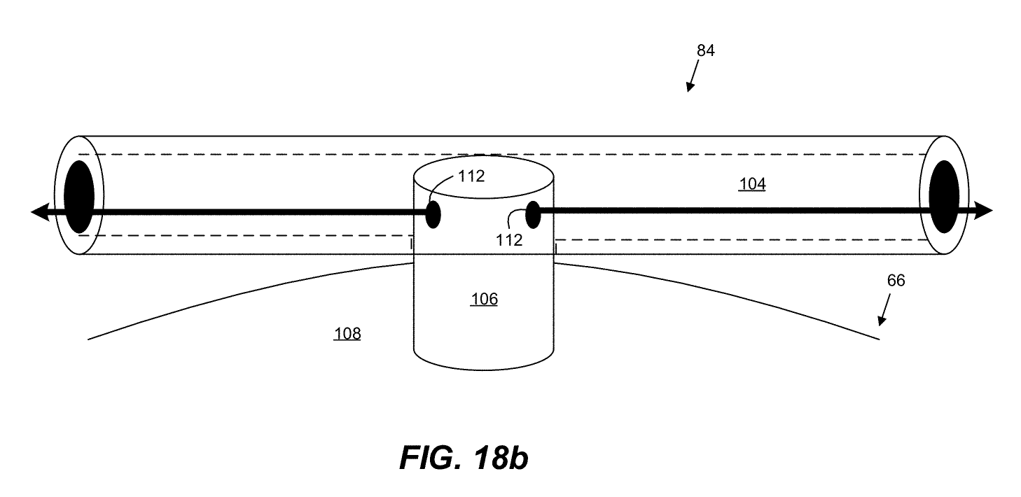

A fourth modality combines the sleep-aid mouthpiece 10 with the external airflow device 64. The external airflow device 64 may be attached to the sleep-aid mouthpiece 10 via, for example, an adapter disposed on the front member 12. As described below in more detail, the external airflow device 64 may introduce air to the mouth through the sleep-aid mouthpiece 10 or to the nostrils through nostril cones 68 or an airflow port 106 that connects to the nasal spreader 84 (FIGS. 18a and 18b). The external airflow device 64 provides positive air pressure to the nose to enhance nose breathing. The fourth modality may be used by individuals who have medium to heavy snoring and/or occasional sleep apnea. It may also be a viable option for non-compliant CPAP customers or other similar positive air pressure products. The external airflow device 64 may open the sinus airways by allowing powered, filtered air to go through larger nostril openings. This may provide better airflow to the back of the mouth via the sinus pathways as well as the combination of the bite blocks 46 and 48 opening the back of the throat. The external airflow device 64 may also be used in combination with the anterior tongue advancement device to further open the back of the throat for less airway obstruction.

In this regard, it should be appreciated that the multi-modal sleep-aid system may provide a flexible solution for improving sleep because the individual has a choice of accessories and may select the modality that works best at any given time.

Having generally described the components and modalities in a multi-modal sleep-aid system, various embodiments of the structure and operation of the sleep-aid mouthpiece 10, the external airflow device 64, and the nasal spreader 84 will be described. FIGS. 1-7 illustrates various embodiments of the sleep-aid mouthpiece 10 that may be comfortably fit in a user's mouth during sleep to promote improved sleep by reducing airway obstruction and improving airflow. The sleep-aid mouthpiece 10 may be particularly useful for individuals who breathe through their mouths at least some of the time while sleeping. The sleep-aid mouthpiece 10 positions the teeth and, in certain embodiments advances the tongue forward, to allow free airflow through the mouth.

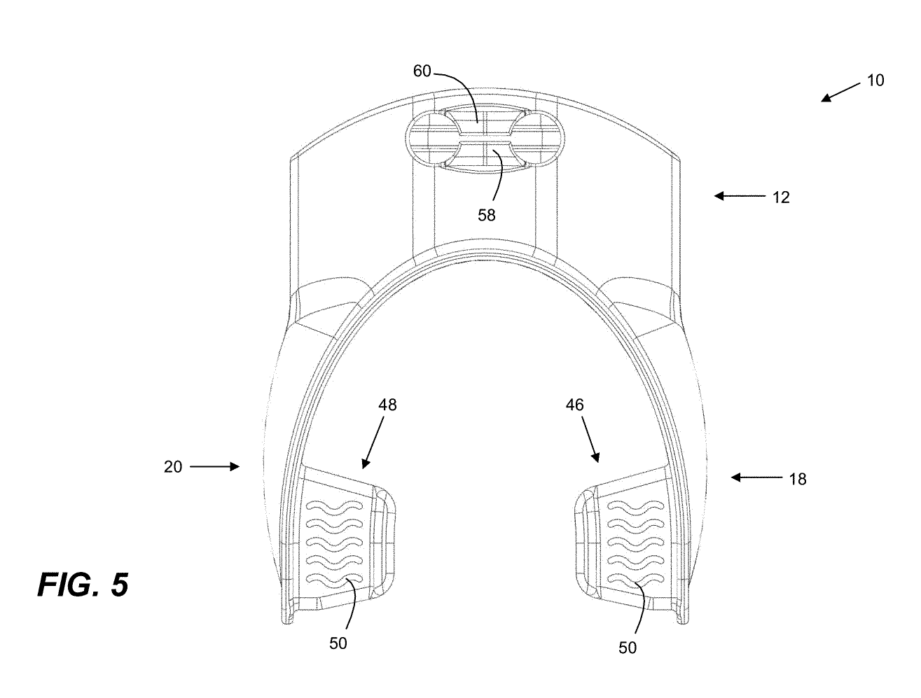

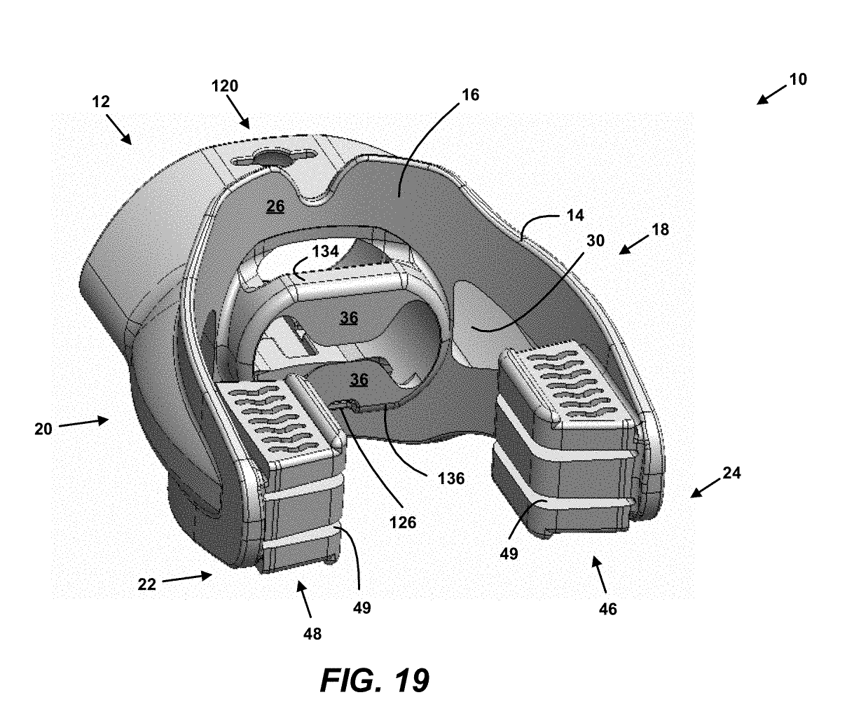

As illustrated in FIG. 1, the sleep-aid mouthpiece 10 comprises a generally U-shaped, in-mouth member 14 configured to be worn in a user's mouth. The U-shaped, in-mouth member 14 defines a middle portion 16 from which a pair of leg portions 18 and 20 extend inwardly to end portions 24 and 22, respectively, inside the user's mouth. The U-shaped, in-mouth member 14 is contoured to conform to fit the lips, helping to keep the mouthpiece 10 in the mouth. The end portions 22 and 24 may support corresponding bite blocks 48 and 46 for reducing teeth grinding. Bite blocks 48 and 46 extend inwardly with an upper surface and a lower surface for supporting the user's rear upper and lower molars. Bite blocks 46 and 48 are disposed to separate the user's teeth, thereby preventing grinding while also opening the mouth and throat for improved airflow while breathing. The upper and/or lower surfaces of the bite blocks 46 and 48 may have ridges 50 (FIG. 5), or other structural projection(s) or recesses, to prevent movement of the user's teeth against the upper and lower surfaces. The physical shape and dimensions of the bite blocks 46 and 48 may be modified in various ways to separate the upper and lower teeth a desirable distance to aid in airflow, as well as, reduce teeth grinding. For example, as illustrated in the alternative embodiment of the sleep-aid mouthpiece in FIG. 19, each of the bite blocks 46 and 48 may be formed by a plurality of detachable block members that are partially separated by recesses or slits 49. In this manner, the height of the bite blocks 46 and 48 may be customized to a desirable height by detaching one or more of the block members.

The sleep-aid mouthpiece 10 further comprises a front member 12 extending from an outer surface 28 of the U-shaped, in-mouth member 14. When worn by the user, the front member 12 is disposed outside of the user's mouth. As best illustrated in FIGS. 1 & 2, in an embodiment, the front member 12 comprises a generally oval-shaped structure. The front member 12 comprises bilateral breathing ports 30 and 32 that define respective air passageways through the front member 12. The air passageways defined by the breathing ports 30 and 32 provide airflow from outside the user's mouth, through the front member 12 and the middle portion 16, and terminating at the inner surface 26 of the U-shaped, in-mouth member 14 inside the user's mouth while being worn.

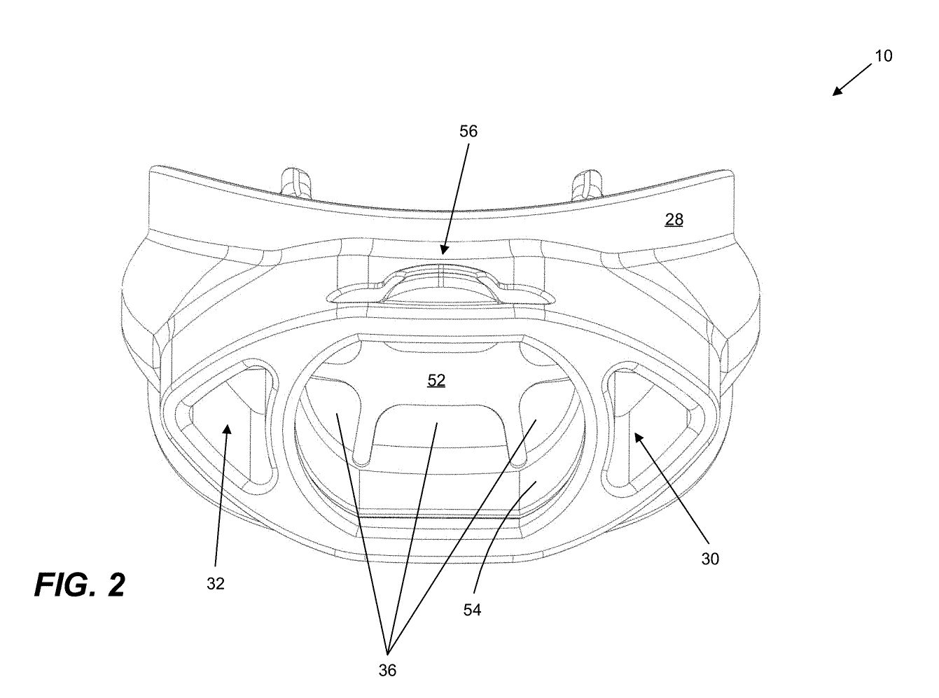

The middle portion 16 of the U-shaped, in-mouth member 14 comprises an anterior tongue advancement device 34 configured to receive and retain the user's tongue in a slightly-forward position to keep the tongue from blocking the user's throat. It should be appreciated that the anterior tongue advancement device 34 may be integrally formed with the middle portion 16 as one or more flexible members 36 surrounding a central passageway 52 (FIG. 2) through the front member 12 to receive the user's tongue. In an embodiment, the flexible member(s) 36 may be symmetrically arranged around the central passageway 52 at the mouth opening. The flexible member(s) 36 hold the user's tongue in place without resulting in stress or fatigue. The flexible members 36 may be slightly angled outward from the mouth and may comprise a tapered arrangement in which the width decreases toward the opening. As illustrated in the embodiment of FIG. 1, the flexible members 36 may comprise six members that generally form the central passageway 52 as a six-pointed star shape in the inner surface 26 of the U-shaped, in-mouth member 12. In this arrangement, the upper and lower members may have a generally rectangular shape, whereas the pair of flexible members on each side of the central passageway 52 defines a pointed or triangular shape. To further assist in breathing when the tongue is retained in the flexible members 36, the middle portion 16 may further comprise one or more additional breath holes (not shown).

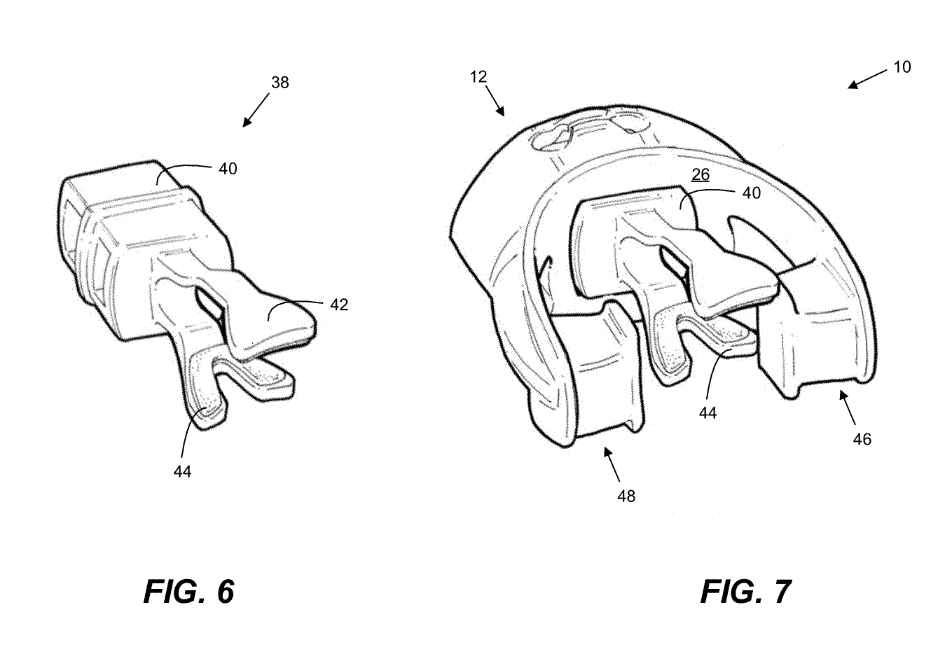

In other embodiments, the central passageway 52 formed in the middle portion 16 of the U-shaped, in-mouth member 12 may be configured to receive a removable anterior tongue advancement device 38 (FIG. 6). In this embodiment, the portion of the central passageway 52 terminating at the inner surface 26 may define a reciprocal structure to receive a similarly-shaped base 40 of the removable anterior tongue advancement device 38. In this regard, the base 40 may be securely inserted into the central passageway 52 to provide an alternative mechanism for receiving and advancing the tongue forward. FIG. 7 illustrates the base 40 inserted into and secured in the central passageway 52. The removable anterior tongue advancement device 38 comprises an upper flexible member 42 and a lower flexible member 44. The upper and lower members 42 and 44 extend inwardly (i.e., into the mouth) from the base 40 to provide an alternative mechanism for receiving and retaining the user's tongue. It should be appreciated that the flexible members 42 and 44 may be arranged in alternative ways and may comprise any number of flexible members or other structures for gently receiving and retaining the user's tongue in a forward position to improve airflow. When the removable anterior tongue advancement device 38 is removed, the central passageway in the middle portion 16 may cooperate with the bilateral breathing ports 30 and 32 to provide additional airflow. In either the integrated or removable embodiment, the anterior tongue advancement device aids in clearing the airway through the mouth and throat, enabling the user to breathe easier.

The front member 12, which extends outside the user's mouth when worn, functions as an adapter mechanism for removably attaching an external airflow device 64. As illustrated in FIGS. 8-13, the external airflow device 64 comprises a housing 66 for containing various components. In an embodiment, the external airflow device 64 may comprise a battery-powered device in which case the housing 66 contains one or more batteries, as well as, a source of airflow. The source of airflow may comprise, for example, a variable-speed motor attached to a lightweight fan. Voltage regulator electronics may be included to maximize the energy utilization of the battery. As known in the art, the battery voltage may not be uniform over time and may be significantly different than that required for optimum operation of the fan. Furthermore, additional electronic components contained in the housing 66 may include a power switch and a microcontroller. The microcontroller may enable or disable power to the fan. Microcontroller firmware may perform various diagnostics and operate a run-time timer. When the timer elapses, the microcontroller may turn off the fan. Sensors may also be included to detect whether the device is being worn. If the device is no longer being worn but is still running, the microcontroller may begin a delay timer. When the timer elapses, the microcontroller may automatically shut off the fan. The housing 66 may further comprise one or more vents for providing air intake for the fan. As described below, further vents 80 and 82 may provide a path for mouth inhalation and/or exhalation air via the adapter connection between the sleep-aid mouthpiece 10 and the housing 66.

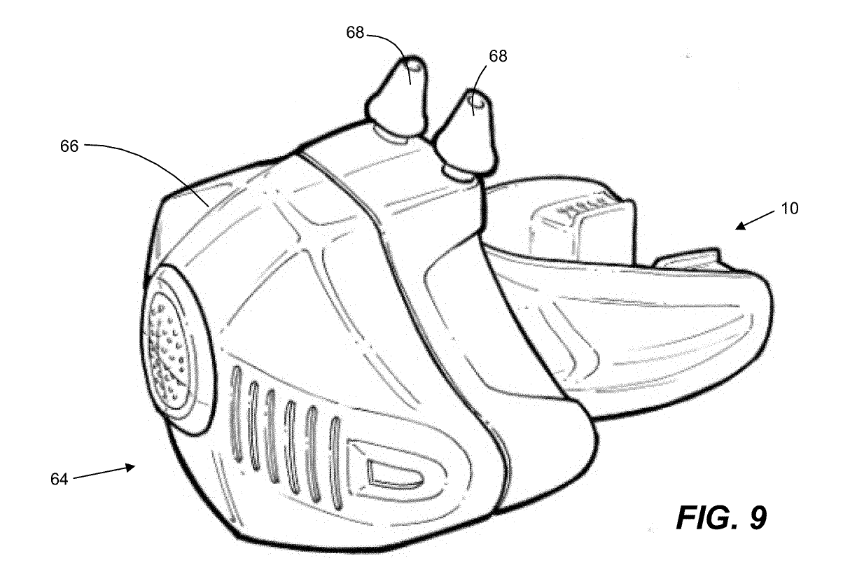

When the external airflow device 64 is attached to the sleep-aid mouthpiece 10 (FIGS. 9, 10b, 11b, and 13), airflow may be introduced from the housing 66 to air passageways within a pair of nostril cones 68. The nostril cones 68 are disposed on the upper surface of the housing 66 such that they may be easily positioned in the user's nostrils when the external airflow device 64 is connected to the sleep-aid mouthpiece 10 and inserted in the user's mouth.

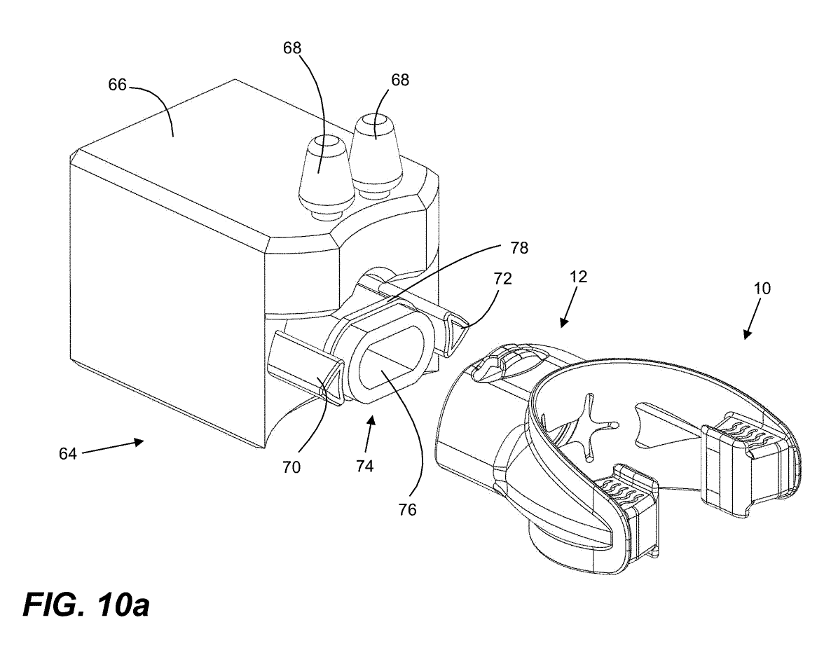

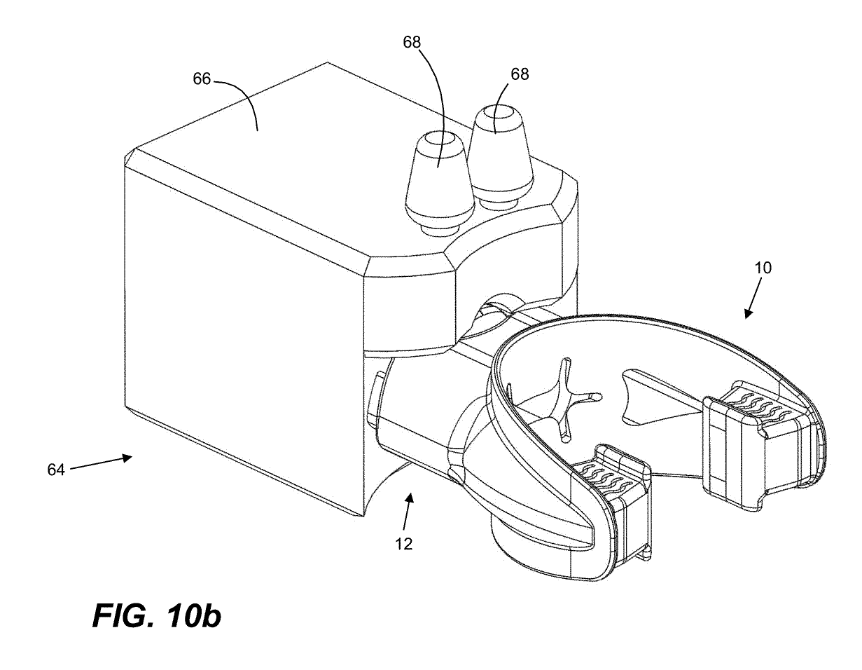

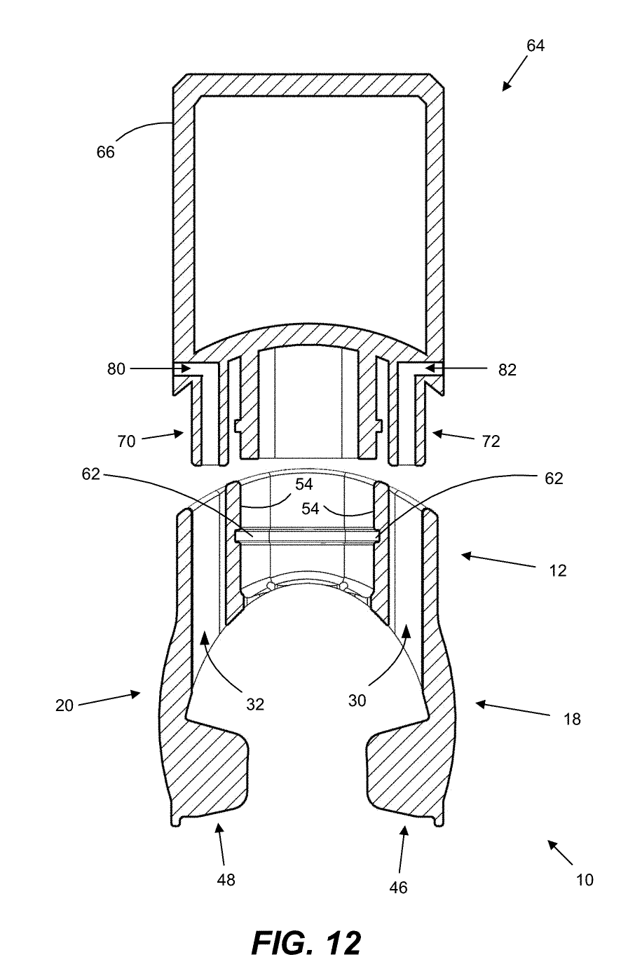

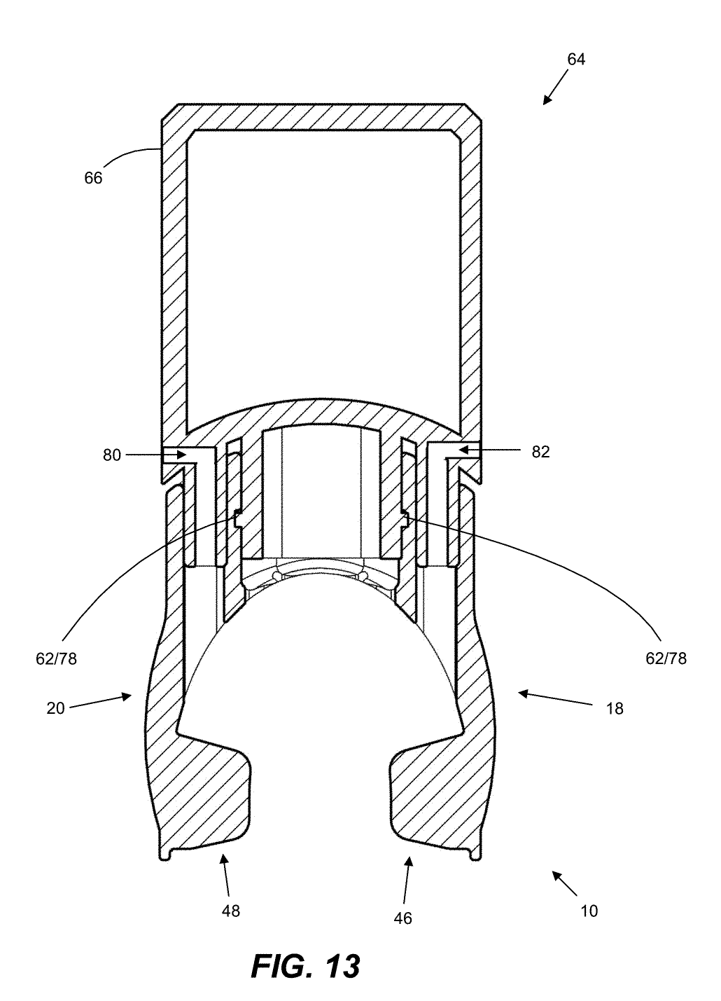

FIG. 10a illustrates an exemplary embodiment of an adapter that may be disposed on the housing 66 of the external airflow device 64 to provide a secure connection to the sleep-aid mouthpiece 10. It should be appreciated that various reciprocal structural members, shapes, etc. may be used to provide the removable attachment of the external airflow device 64 to the front member 12 of the sleep-aid mouthpiece 10. In the embodiment of FIG. 10a, the adapter mechanism comprises structural components to be received within the bilateral breathing ports 30 and 32 and the central passageway 52 disposed on the front member 12. Bilateral members 70 and 72, which extend away from the housing 66, may be inserted into the bilateral breathing ports 30 and 32. As illustrated in FIGS. 12 and 13, the bilateral members 70 and 72 may have channels or passageways for providing airflow through the bilateral members 70 and 72 and venting at ports 80 and 82. In this manner, when the external airflow device 64 is attached to the sleep-aid mouthpiece 10 (FIG. 13), the user may inhale and exhale through the bilateral breathing ports 30 and 32 and the corresponding port 80 and 82. As further illustrated in FIG. 10a, the external airflow device 64 may comprise a central member 74 for providing additional support and connection to the sleep-aid mouthpiece 10. The central member 74 may be configured to be received within the central passageway 52 in the front member 12 of the sleep-aid mouthpiece 10.

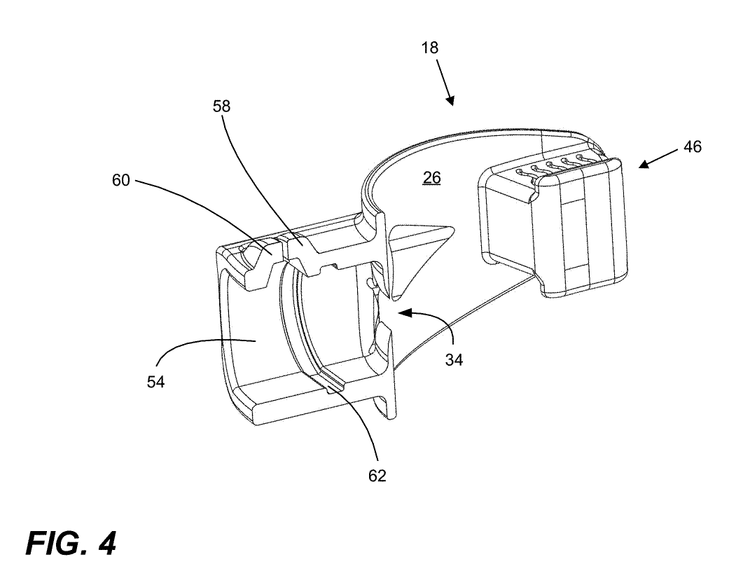

In an embodiment, the central member 74 is generally oval-shaped to be received within the oval-shaped central passageway 52 (FIG. 2) in the front member 12. To provide a more secure attachment, the outer surface of the oval-shaped central member 74 on the external airflow device 64 may include a key/keyway 78 to be secured with a cooperating key/keyway 62 (FIG. 4, 11a) on an inner wall 54 of the front member 12.

In this regard, the sleep-aid mouthpiece 10 provides three separate points of contact with the external airflow device 64 for providing a more stable connection. A first point of contact comprises the central member 74 on the external airflow device 64 securely received in the inner wall 54 of the sleep-aid mouthpiece 10. The second and third contact points comprise the bilateral members 70 and 72 on the external airflow device 64 securely received in the breathing ports 32 and 30, respectively. It should be appreciated that this three-point structural integration provided by the adapter mechanism between the sleep-aid mouthpiece 10 and the external airflow device 64 provides a stable, secure connection during use that may reduce physical stress on the user while sleeping. Furthermore, the external airflow device 64 may support further stabilization means, such as, for example, one or more connections for attaching a headstrap. The multi-modal sleep-aid system may also leverage additional points of contact for further support when the nasal spreader 84 and/or the nostril cones 68 are included. For example, a connection between the nostril cones 68 disposed on the external airflow device 64 and the user's nostrils may provide another point of contact for further support during use. Depending on the particular modality, a connection between the nasal spreader 84 and the sleep-aid mouthpiece 10 or the external airflow device 64 may also provide another point of contact for stress-preventing support and comfort during use.

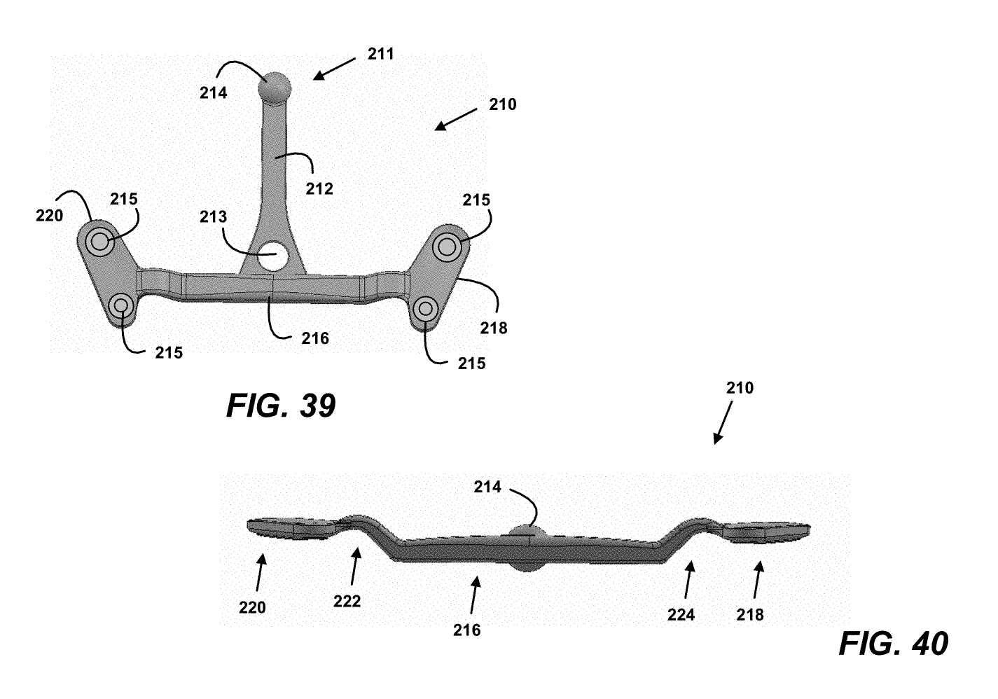

As mentioned above, in certain modalities, the sleep-aid mouthpiece 10 may be combined with a nasal spreader accessory 84 or a tether nasal spreader accessory 210 (as described below in connection FIGS. 39 & 40). In this regard, the sleep-aid mouthpiece 10 may comprise a nasal spreader attachment mechanism 56 disposed on, for example, a middle portion of an upper surface of the front member 12. As illustrated in the embodiment of FIG. 4, the nasal spreader attachment mechanism 56 comprises a latch having a pair of opposing bar members 58 and 60. Bar members 58 and 60 define a recess or channel in which a nasal spreader accessory 84 may be secured. FIG. 14 illustrates an exemplary nasal spreader 84 secured by the latch defined by bar members 58 and 56.

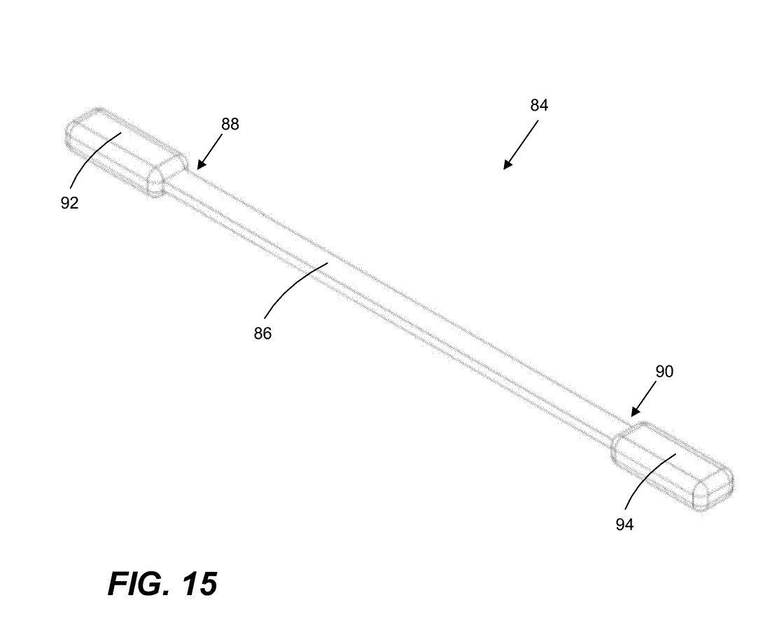

It should be appreciated that the latching mechanism may be implemented in various alternative ways to securely attach the nasal spreader 84 to the sleep-aid mouthpiece 10. FIGS. 14 and 15 illustrate one of a number of possible configurations for the nasal spreader 84. In general, the nasal spreader 84 comprises a flexible elongate member 86 with opposing ends 88 and 90 having nostril pads 92 and 94, respectively. The nostril pad 92 and 94 are adapted to be inserted into the user's nostrils when the sleep-aid mouthpiece 10 is worn in the user's mouth. In an embodiment, the flexible elongate member 86 comprises a material which is reversibly deformable from a straight default state (FIG. 15) to a flexed state (FIG. 14) in which the respective nostril pads 92 and 94 on the first and second ends 88 and 90 may be inserted into the user's nostrils. When inserted in a user's nostrils in the flexed state, the ends 88 and 90 of the flexible elongate member 86 apply a desirable force to comfortably expand the user's nostrils and open the nasal airways. The table below illustrates measurements for the applied force at a 1-inch spread measured at initial flex for various length, width, and thickness values for the flexible elongate member 86.

| Force @ 1″ spread | | | |
|---|---|---|---|
| measured at initial flex | Length | Width | Thickness |
| (ounces) | (inches) | (inches) | (inches) |
| 0.90 oz | 3.35 | 0.096 | 0.036 |
| 1.30 oz | 3 | 0.096 | 0.036 |
| 1.50 oz | 2.5 | 0.096 | 0.036 |
| 1.80 oz | 4.5 | 0.136 | 0.052 |
| 1.80 oz | 2 | 0.096 | 0.036 |
| 2.00 oz | 4.5 | 0.1875 | 0.047 |
| 2.50 oz | 4 | 0.136 | 0.052 |
| 2.70 oz | 3.5 | 0.136 | 0.052 |
| 3.50 oz | 3.75 | 0.1875 | 0.047 |
| 3.50 oz | 3.5 | 0.1875 | 0.047 |
| 3.50 oz | 3 | 0.136 | 0.052 |
| 5.00 oz | 2.5 | 0.136 | 0.052 |
| 5.90 oz | 2 | 0.136 | 0.052 |

In an exemplary embodiment, the flexible elongate member 86 may be formed from a plastic material, such as, Nylon (e.g., Nylon 66) or other desirable flexible plastics or other materials. The nostril pads 92 and 94 may be formed from a soft material, such as, the material used in first aid tape (e.g., 3M Nexcare™ First Aid Tape), which may be applied to the ends 88 and 90 in liquid form or otherwise. It should be appreciated that the dimensions of and the materials used for the nasal spreader 84 may be adapted to accommodate various individual preferences and sizes.

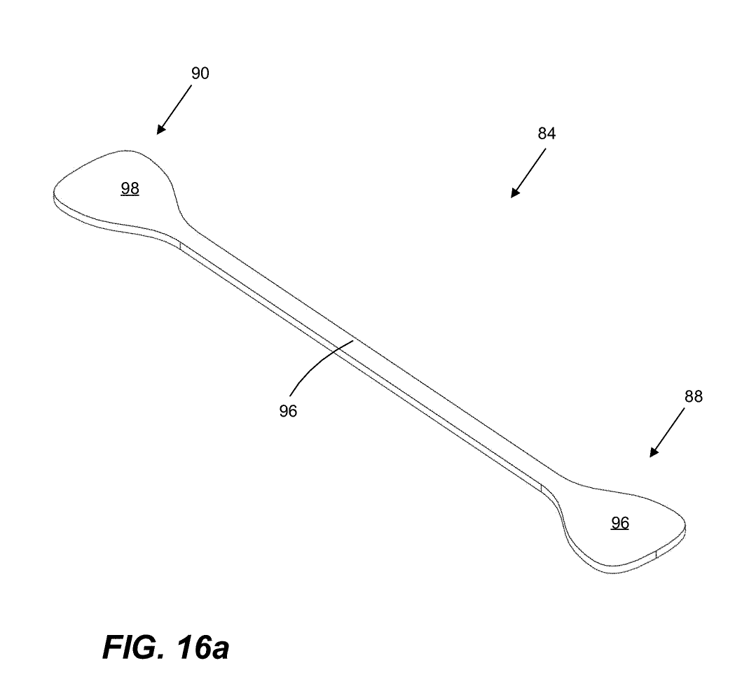

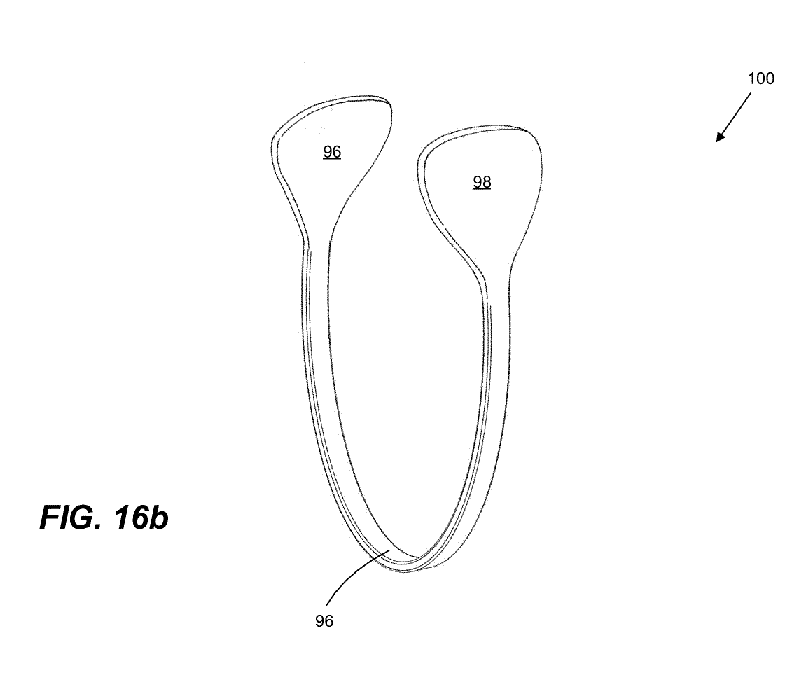

As best illustrated in FIG. 15, the flexible elongate member 86 comprises a strip having a rectangular cross-section. The nostril pads 92 and 94 may comprise separate pads that are fixed to the ends 88 and 90. In other embodiments, the ends 88 and 90 may comprise nostril members 98 and 96 that are integrally formed with a rectangular flat member 97. It should be appreciated that the nostril members 98 and 96 may comprise any desirable shape that comfortably fits within the user's nostrils and expands the nostrils to improve airflow. In the embodiment of FIG. 16, the nostril member 98 and 96 comprise a flat tear-shaped member having a same height as the rectangular flat member 97.

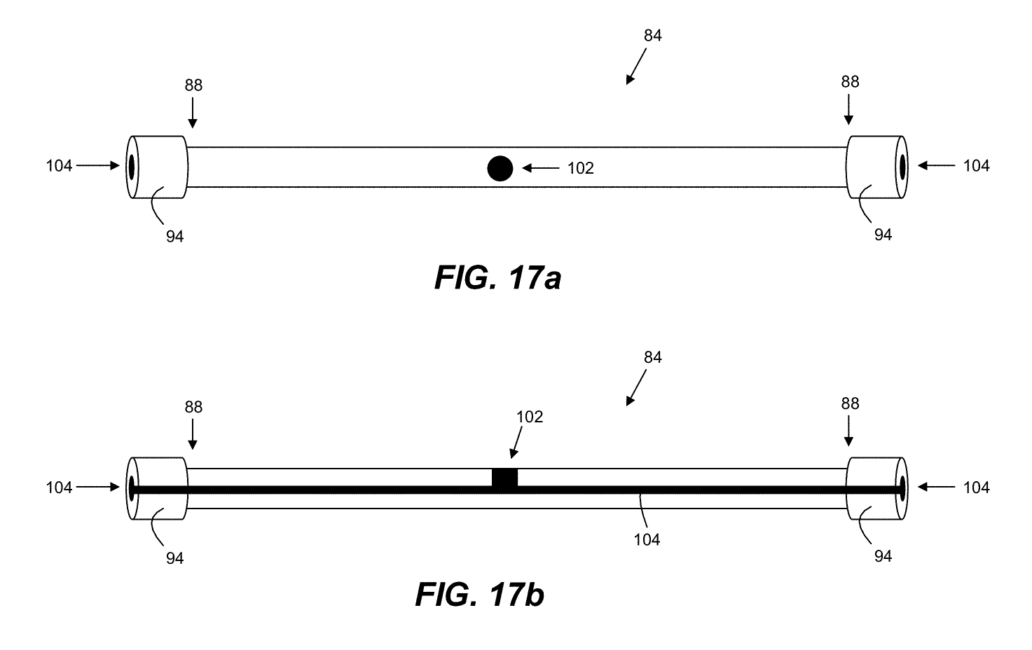

FIGS. 17a and 17b illustrate another embodiment of the nasal spreader accessory 84. In this embodiment, the flexible elongate member comprises a longitudinal bore 104 extending a length of the member and through the respective nostril members 94 and 92. The nasal spreader 84 further comprises one or more airflow ports 102 disposed on a middle portion of the flexible elongate member 86. The airflow ports 102 may be used to introduce airflow from the external airflow device 64 into the longitudinal bore 104 and into the user's nostrils.

As illustrated in FIGS. 18a and 18b, the external airflow device 64 may include an airflow port 106 that generally extends upward from an upper portion 108 of the housing 66 to allow for connection to an airflow port 102 disposed on the middle portion of the flexible elongate member 86. In an embodiment, the airflow port 106 may comprise a hollow, cylindrically shaped member. The hollow, cylindrically shaped member is sized to securely fit in the airflow port 102 on the nasal spreader 84. The airflow port 106 may include one or more holes 112 disposed adjacent an outer end 110 where it mates with the nasal spreader 84. The outer end 110 may be inserted into the airflow port 102 of the nasal spreader 84 such that the holes 112 are positioned within the longitudinal bore 104. In this manner and as illustrated in FIG. 18b, the air produced by the external airflow device 64 flows into the hollow, cylindrically shaped member and through the holes 112 into the longitudinal bore 104. The air flows through the longitudinal bore 104, exits the nasal spreader 84 at the nostril pads 92 and 94, and flows into the nostrils. FIG. 18b illustrates the nasal spreader 84 in the straight default state prior to being deformed to the flexed state for insertion in the nostrils. It should be appreciated that the outer end 110 may comprise a press-fit connection to the airflow port 102. In other embodiments, the nasal spreader 84 may comprise a protruding tube section for mating with the hollow cylindrically shaped member, in which case the hole(s) 112 may be disposed on a top surface of the outer end 110 rather than on the sides of the cylinder. In further embodiments, the nasal spreader 84 may include an adapter for quickly, easily and securely connecting the nasal spreader 84 to the external airflow device 64.

FIG. 19 is a perspective view of another embodiment of a sleep-aid mouthpiece 10, which may be used in the multi-modal sleep-aid systems and methods described above in connection with FIG. 1-18. As illustrated in FIG. 19, the sleep-aid mouthpiece 10 comprises another embodiment of an integral tongue advancement device 34. In this embodiment, the tongue advancement device 34 extends inward away from the inner surface 26 of the U-shaped, in-mouth member 14. It should be appreciated that, by extending inward, this configuration may provide easier interaction with the user's tongue.

As best illustrated in the embodiment of FIGS. 19 & 21, the inwardly-extending tongue advancement device 34 may generally form an oval-shaped member defined by side portions 130 and 132, a top member 134, and a bottom member 136. The top member 134 may comprise an opening 128 for increasing airflow through the central passageway 52. Opposing upper and lower flexible member(s) 36 may be arranged to receive and hold the user's tongue in place without resulting in stress or fatigue. The upper and lower flexible members 36 may define figure-eight shaped lateral openings. To further assist in breathing when the tongue is being retained, the flexible members 36 may be comprise one or more holes. As illustrated in FIG. 20, an outer surface of the flexible members 36 may comprise one or more circular or other-shaped cut-outs 127 to reduce stress or fatigue.

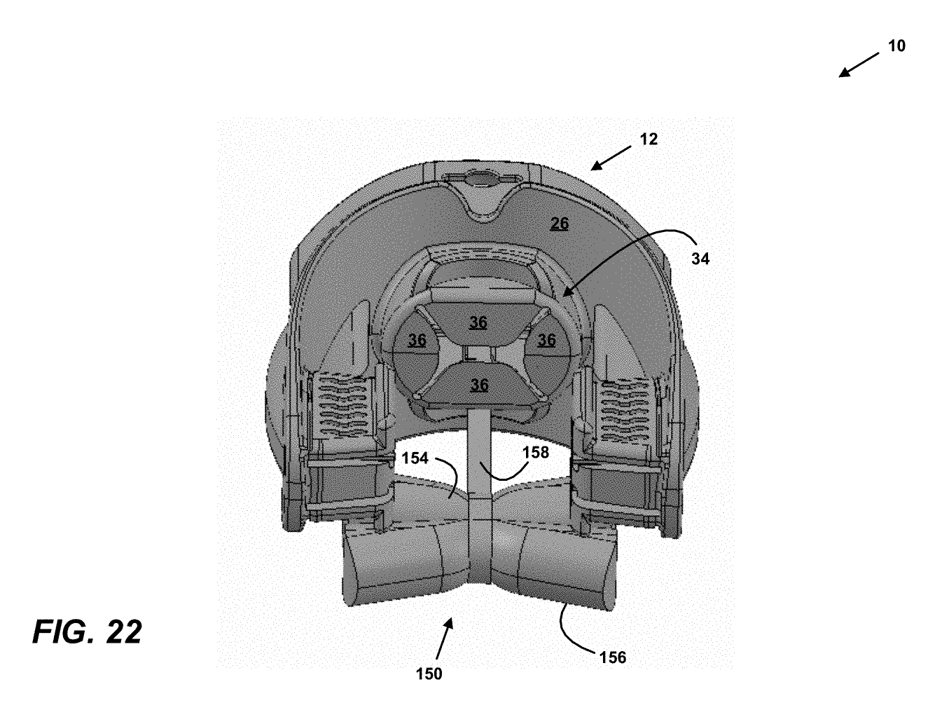

As mentioned above, the U-shaped, in-mouth member 14 may be contoured to conform to fit the lips, helping to keep the mouthpiece 10 in the mouth. In the embodiment of FIG. 22, a middle portion of the surfaces 26 and 28 may include a curved cut-out 122 for improved comfort.

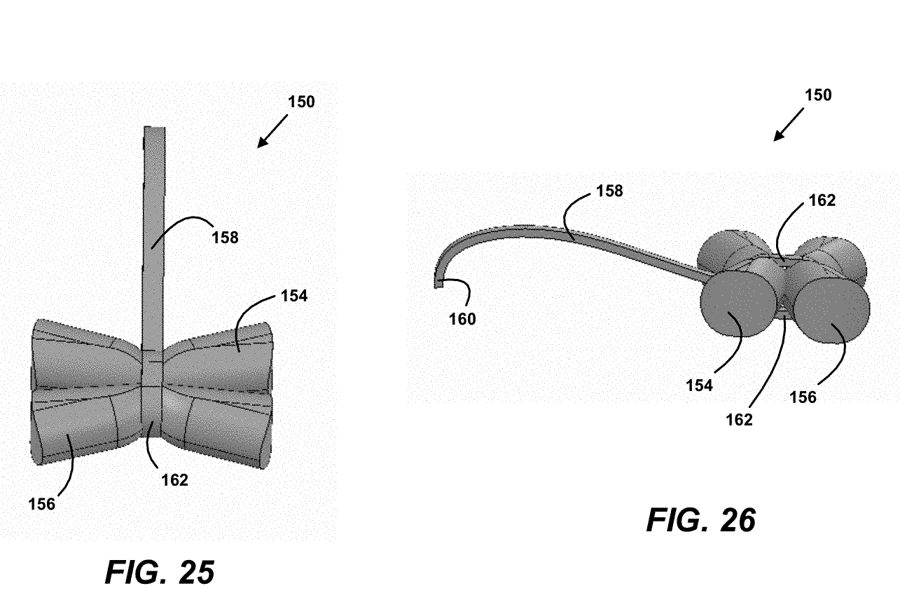

The sleep-aid mouthpiece 10 may be further configured to cooperate with an optional anti-drool accessory. An embodiment of an anti-drool accessory 150 is illustrated in FIGS. 25 and 26. The anti-drool accessory 150 comprises a flexible elongate member 158 secured to a sterile absorbent material, such as, for example one or more sterile cotton rolls 154 and 156. In one example, the flexible elongate member 158 may comprise a sterilizable self-locking cable tie having a portion 162 that may be secured around the one or more sterile cottons 154 and 156, as illustrated in FIG. 26, and fixed in place.

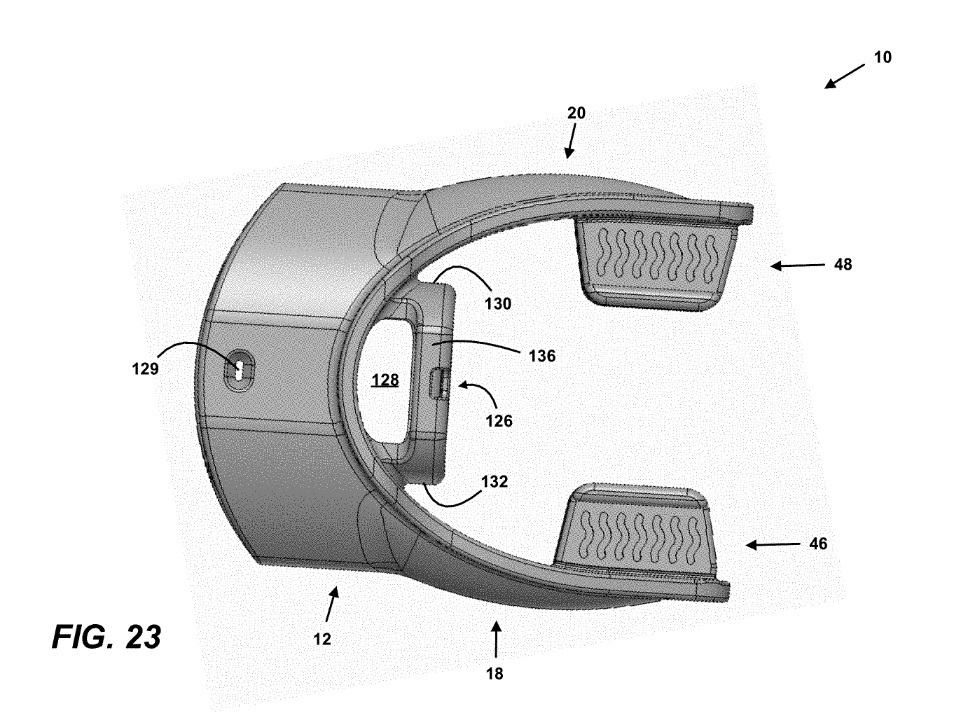

To accommodate optional use with the anti-drool accessory 150, the sleep-aid mouthpiece 10 may be configured with an opening, channel, and/or other alignment/retention features. As best illustrated in FIGS. 19, 20, 23, 24, and 27, the bottom member 136 of the tongue advancement device 34 may comprise an opening 126 for inserting and receiving the end of the flexible elongate member 158 of the anti-drool accessory 150. Furthermore, a lower portion of the inner wall 54 defining the central passageway 52 in the front member 12 may comprise a channel 124 for aligning and receiving the flexible elongate member 168 as it is installed. FIGS. 22 & 24 illustrate the anti-drool accessory 150 installed in the sleep-aid mouthpiece 10. As best illustrated in FIG. 23, a lower portion of the front member 12 may include an additional opening 129 for receiving the end 160 of the flexible elongate member 158 when the anti-drool accessory 150 is installed and secured in place. When the anti-drool accessory 150 is installed and the sleep-aid mouthpiece 10 is properly inserted in the user's mouth, the sterile cotton rolls 154 and 156 may be easily positioned below the user's tongue to absorb saliva and reduce drooling.

In this regard, it should be appreciated that the sleep-aid mouthpiece 10 may advantageously provide a plurality of contact points for safely securing the anti-drool accessory 150 to the sleep-aid mouthpiece 10 while in use. The opening 126 in the anterior tongue advancement device 124 may provide a first point of contact. A second point of contact may comprise the channel 124 formed in the inner wall 54 of the front member 12. A third point of contact may comprise the opening 129 disposed on the lower portion of the front member 12 for receiving the end 160.

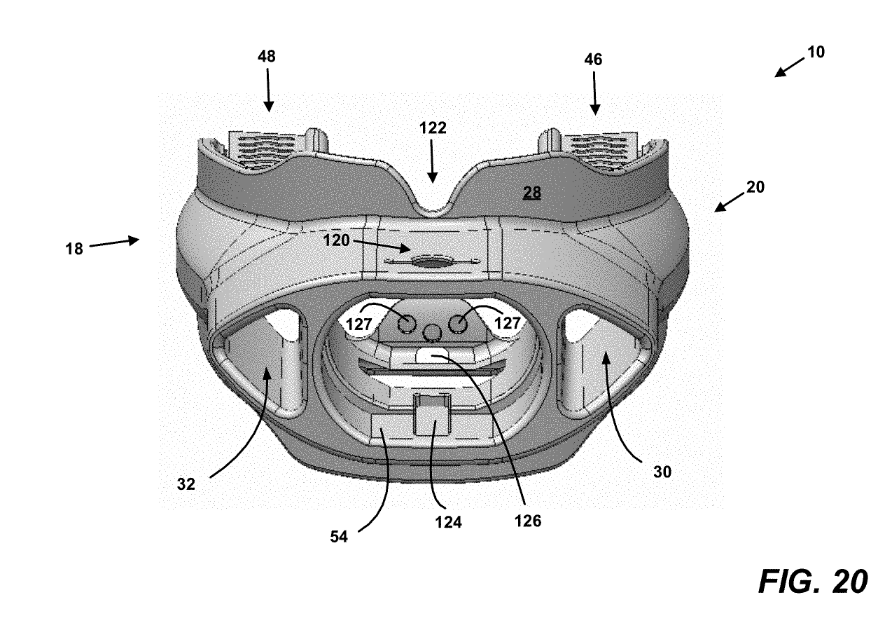

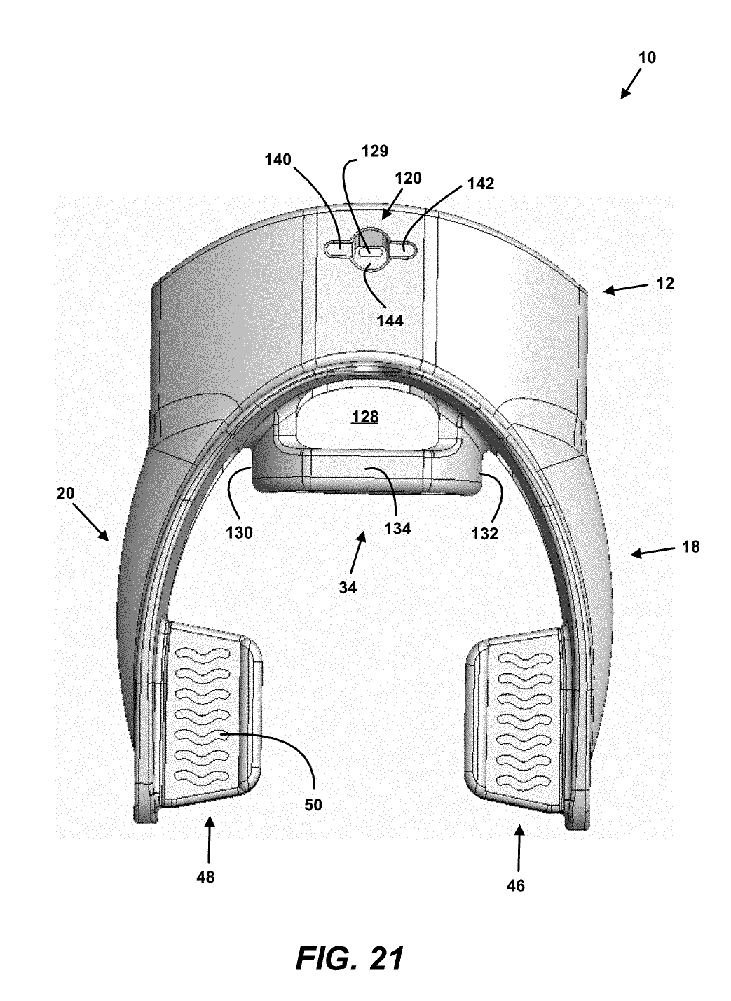

In the embodiment of FIG. 19, the sleep-aid mouthpiece 10 comprises another embodiment of a nasal spreader attachment mechanism 120 disposed on, for example, a middle portion of an upper surface of the front member 12. As best illustrated in FIG. 21, the nasal spreader attachment mechanism 120 comprises a circular opening 144 with opposing slits 140 and 142. The nasal spreader attachment mechanism 120 may be a desirable configuration for securing a ball-type nasal spreader accessory. As illustrated in the embodiment of FIGS. 39 & 40, an exemplary ball-type nasal spreader accessory 210 comprises a flexible, T-shaped member defined by transverse elongate members 212 and 216. A retaining ball member 214 may be disposed on one end 211 of elongate member 212. The retaining ball member 214 is sized to be inserted and securely received in the circular opening 144 in the nasal spreader attachment mechanism 120. Each end of the flexible elongate member 216 comprises a flat paddle 218 and 220. Paddles 218 and 220 may be angled away from transverse elongate member 212. As further illustrated in FIG. 40, the flat paddles 218 and 220 may be raised relative to the elongate member 216 via angled connecting members 224 and 222, respectively. A surface of the flat paddles 218 and 220 may include visual guides (e.g., circular trim ridges or recesses 215) forming a reference template for reducing the size of the paddles by cutting along the visual guides. In the embodiment of FIG. 39, each paddle 218 and 220 comprises a pair of concentric trim ridges or recesses 215 disposed on respective ends. In use (either independently or in combination with the sleep-aid mouthpiece 10), the elongate member 216 may be flexed, and then the flat paddles 218 and 220 inserted into the user's nostrils. It should be appreciated that, in use, the flat paddles 218 and 220 may expand the user's nostrils and improve nasal airflow. It should be further appreciated that the tethered nasal spreader accessory 210 may be formed from two different materials. The elongate member 212 comprising the tether to the sleep-aid mouthpiece 10 may be formed from a flexible material, whereas the elongate member 216 may be formed from a more rigid second material. As described above in connection with nasal spreader 84, the second material may be reversibly deformable from a relatively straight default state to a flexed state in which the respective paddles 218 and 220 may be inserted into the user's nostrils. When inserted in a user's nostrils in the flexed state, the paddles 218 and 220 formed on the ends of the elongate member 216 apply a desirable force to comfortably expand the user's nostrils and open the nasal airways.

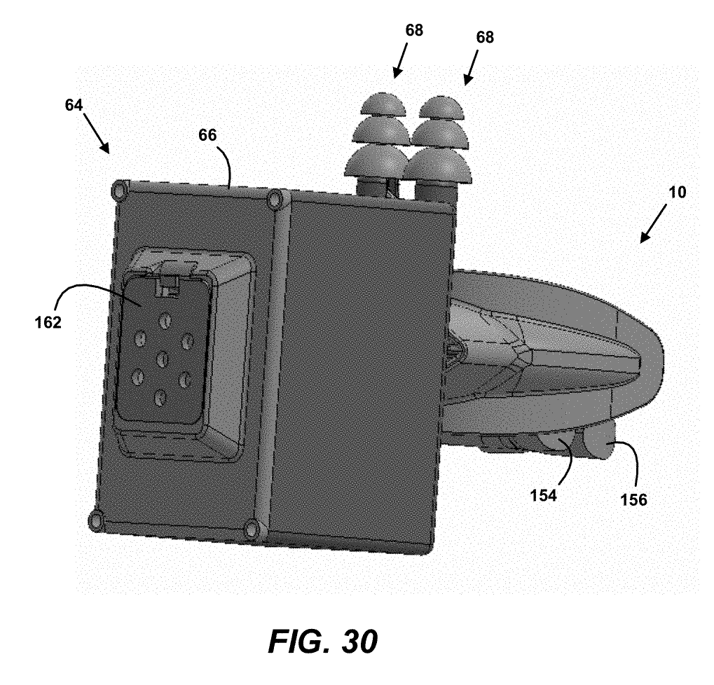

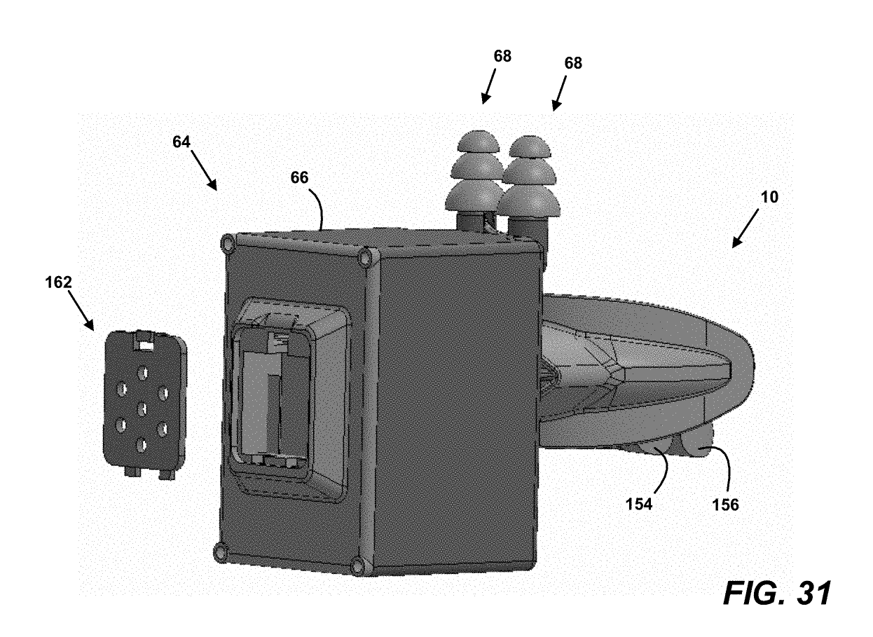

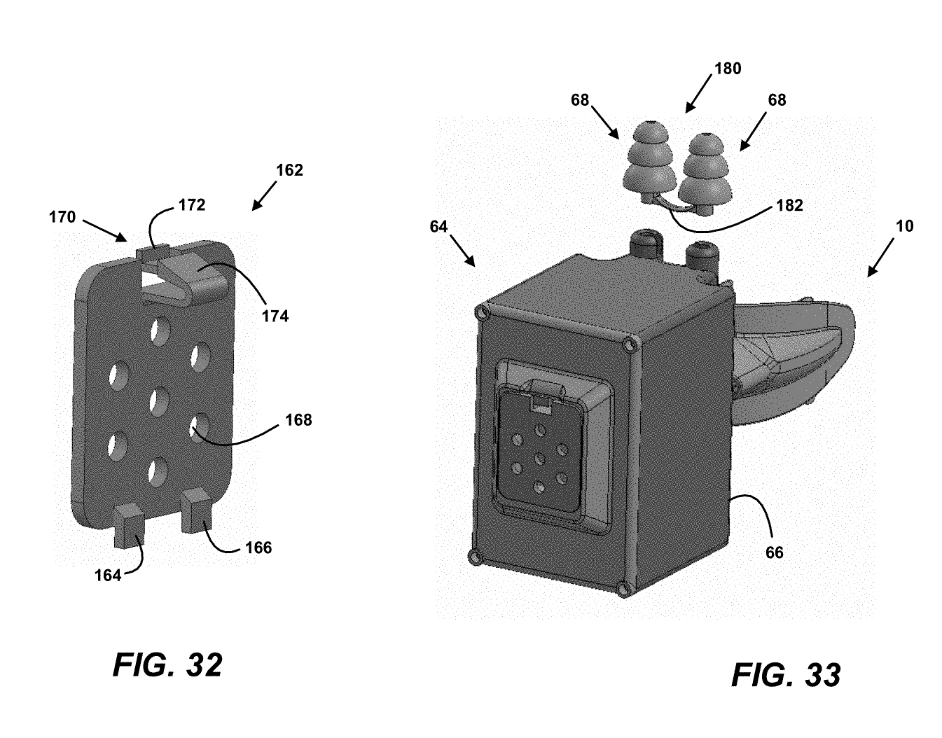

Another embodiment of the external airflow device 64 is illustrated in FIGS. 30-38. As mentioned above, the external airflow device 64 may introduce air to the mouth through the sleep-aid mouthpiece 10 or to the nostrils through nostril cones 68 or an airflow port 106 that connects to the nasal spreader accessory 84. In this alternative embodiment, the housing 66 comprises an air filter cover 162 and a baffled nostril cone configuration. The air filter cover 162 may be easily removed (FIG. 31) to provide convenient access to a recess within the housing 64 for replacing an air filter. As known in the art, the removable air filter may comprise fibrous material(s) for removing solid particulates such as dust, pollen, mold, bacteria, etc. the air. In an embodiment, the air filter may be easily installed and/or removed via the air filter cover 162 and advantageously refrigerated and/or treated with, for example, mentholated liquid(s), material(s), etc. to provide a cooling effect that may further open nasal airways. As illustrated in the embodiment of FIG. 32, the air filter cover 162 comprises securing members 164 and 166 and a spring-type release/locking mechanism 170. The securing members 164 and 166 may be inserted into corresponding recesses within the housing 64. The release/locking mechanism 170 comprises a curved member 174 for providing the spring force, and a latch 172 for engaging with a corresponding keyway in the housing 164. In additional embodiments, the air filter cover may comprise alternative mechanisms for detaching and securing to the housing 66. For example, the air filter cover 162 may be removably attached using a sliding/locking configuration or means other than the release/locking mechanism 170.

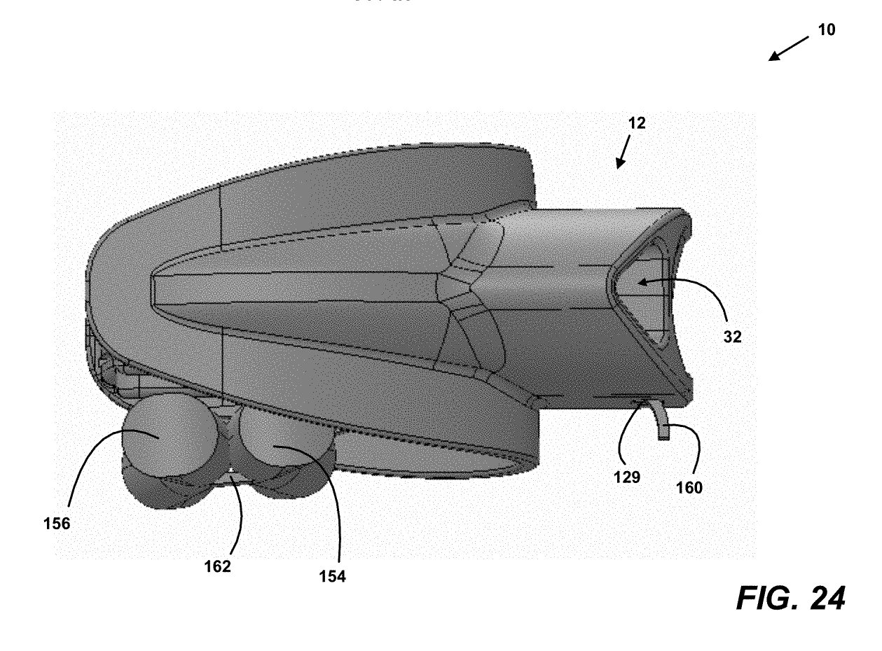

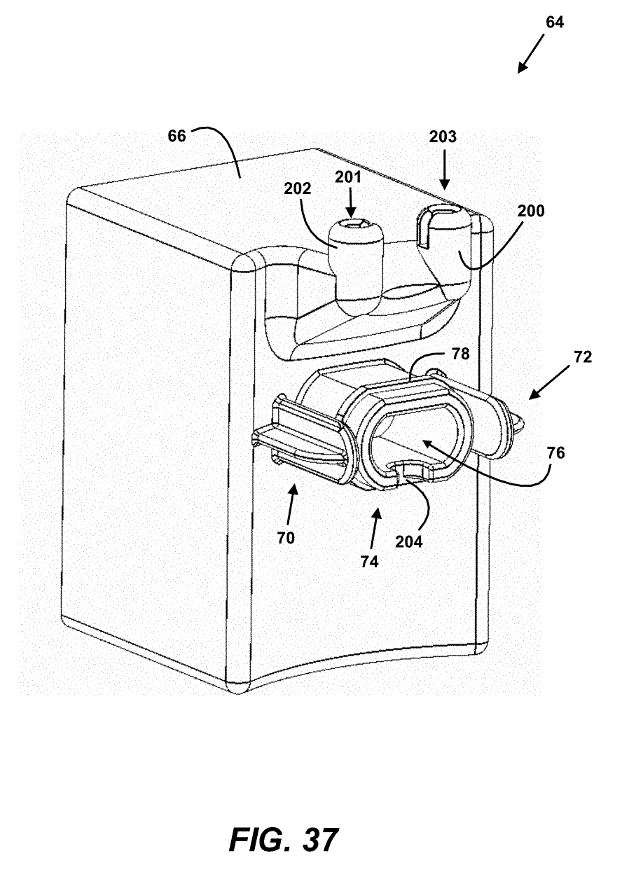

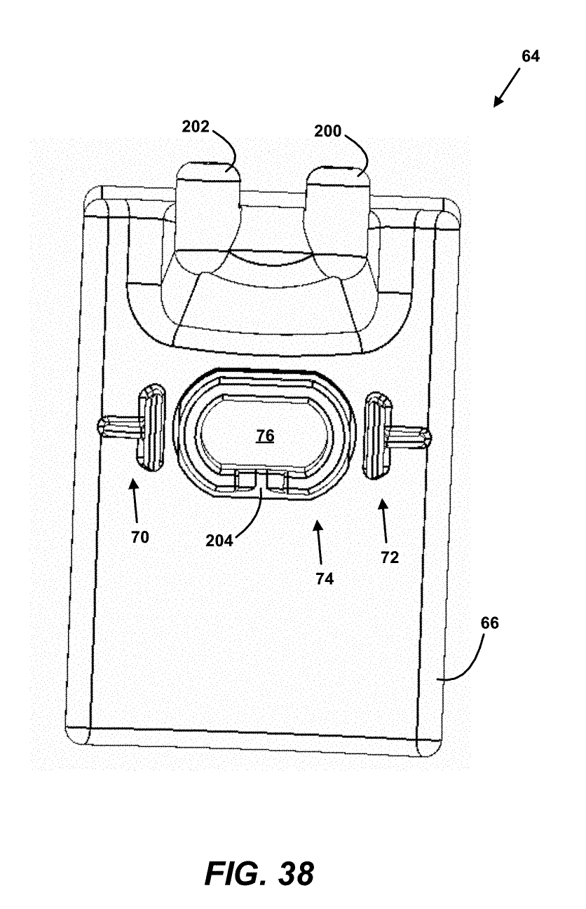

FIGS. 37 & 38 illustrates another embodiment of an adapter mechanism for securing to the sleep-aid mouthpiece. In this embodiment, the bilateral members 70 and 72, which extend away from the housing 66 and are inserted into the bilateral breathing ports 30 and 32, comprise a T-shaped cross-sectional structure. Unlike the embodiment described above in connection with FIGS. 10-13, the bilateral members 70 and 72 may not necessarily include channels or passageways for providing airflow through the bilateral members 70 and 72 and venting at ports 80 and 82. As best illustrated in FIG. 38, the central member 74 may further comprise a channel 204 configured to receive and/or secure the end 160 protruding through the opening 129 in the sleep-aid mouthpiece (FIG. 24).

FIGS. 33-36 illustrates an exemplary baffled nostril cones accessory 180. The accessory 180 comprises a pair of baffled nostril cones 68 connected via an attachment member 182. Each nostril cone 68 comprises three baffles 184, 186, and 188 with increasing size to provide easy insertion into the user's nostrils. As illustrated in FIGS. 35 & 36, each baffled nostril cone 68 comprises an airflow passageway 90 therethrough. When installed on corresponding airflow ports 200 and 202 disposed on the housing 66, the external airflow device 64 may introduce positive air pressure through the airflow passageways 90.

Alternative embodiments will become apparent to one of ordinary skill in the art to which the invention pertains without departing from its spirit and scope. Therefore, although selected aspects have been illustrated and described in detail, it will be understood that various substitutions and alterations may be made therein without departing from the spirit and scope of the present invention, as defined by the following claims.

What is claimed is:

1. A sleep-aid mouthpiece comprising:
a U-shaped, in-mouth member configured to be worn in a user's mouth, the U-shaped, in-mouth member comprising a middle portion from which a pair of leg portions extend inwardly to respective end portions inside the user's mouth;
a front member extending from an outer surface of the U-shaped, in-mouth member and disposed outside of the user's mouth;
a pair of lateral breathing ports extending from outside the user's mouth through the front member and the U-shaped, in-mouth member and into the user's mouth; and
an anterior tongue advancement device disposed on an inner surface of the middle portion of the U-shaped, in-mouth member, the anterior tongue advancement device comprising one or more flexible members for receiving and holding the user's tongue in a forward position.

2. The sleep-aid mouthpiece of claim 1, wherein the anterior tongue advancement device is removably secured into an opening on the inner surface of the middle portion of the U-shaped, in-mouth member.

3. The sleep-aid mouthpiece of claim 1, wherein the one or more flexible members are integrally formed from the inner surface of the middle portion of the U-shaped, in-mouth member.

4. The sleep-aid mouthpiece of claim 1, wherein a front portion of the front member comprises an adapter for removably attaching an external airflow device.

5. The sleep-aid mouthpiece of claim 4, wherein the adapter comprises a central passageway having an inner wall for receiving a reciprocal adapter member disposed on the external airflow device.

6. The sleep-aid mouthpiece of claim 1, further comprising a pair of bite blocks disposed on the respective end portions of the leg portions.

7. The sleep-aid mouthpiece of claim 1, further comprising a nasal spreader attachment mechanism disposed on an upper surface of the front member for removably securing a nasal spreader accessory.

8. The sleep-aid mouthpiece of claim 1, wherein a front portion of the front member comprises a central passageway extending through the front member and the U-shaped, in-mouth member, the central passageway having a first end and a second end, the first end defined by an inner wall disposed on the front portion of the front member for removably attaching an external airflow device, the second end defined by the one or more flexible members of the anterior tongue advancement device.

9. The sleep-aid mouthpiece of claim 1, wherein the anterior tongue advancement device extends away from the inner surface of the middle portion of the U-shaped, in-mouth member.

10. The sleep-aid mouthpiece of claim 1, wherein the anterior tongue advancement device comprises an opening for receiving an anti-drool device.

11. The sleep-aid mouthpiece of claim 10, wherein the inner wall disposed on the front portion of the front member comprises one of a channel and a further opening for receiving the anti-drool device.

12. The sleep-aid mouthpiece of claim 11, wherein the anti-drool device comprises a sterilizable elongate member having a first end configured to be received in the opening in the anterior tongue advancement device and the channel or the further opening in the inner wall disposed on the front portion of the front member, and a second end fixed to an absorbent material positionable under the user's tongue.

13. A portable airflow accessory for a sleep-aid mouthpiece, the portable airflow accessory comprising:
a housing containing a source of airflow powered by a battery; and
an adapter disposed on the housing for removably securing to a sleep-aid mouthpiece, the adapter comprising:
a pair of lateral members configured to be removably secured in a pair of corresponding lateral breathing ports disposed on the sleep-aid mouthpiece; and
a center securing member configured to be removably secured in a central passageway disposed on the sleep-aid mouthpiece.

14. The portable airflow accessory of claim 13, wherein the center securing member comprises one of a key and a keyway disposed on an outer surface to be received by the other of the key and keyway disposed on an inner surface defined by the central passageway disposed on the sleep-aid mouthpiece.

15. The portable airflow accessory of claim 13, wherein the pair of lateral members provide airflow through corresponding vents.

16. The portable airflow accessory of claim 13, further comprising:
one or more airflow ports disposed on an upper portion of the housing and in communication with the source of airflow.

17. The portable airflow accessory of claim 16, wherein the one or more airflow ports are adapted to be fixed to one or more corresponding nostril cones configured for insertion in the user's nostrils while the user is wearing the sleep-aid mouthpiece and the adapter is removably secured to the sleep-aid mouthpiece.

18. The portable airflow accessory of claim 17, wherein the one or more nostril cones comprise a baffled nostril cone.

19. The portable airflow accessory of claim 13, wherein the center securing member comprises an oval-shaped tube having an outer surface.

20. The portable airflow accessory of claim 19, wherein the outer surface of the oval-shaped tube comprises one of a key member and a keyway member adapted to be received by the other of the key member and the keyway member disposed on a surface of the central passageway on the sleep-aid mouthpiece.

21. The portable airflow accessory of claim 13, wherein the housing comprises a removable air filter cover.

22. A sleep-aid system comprising:
a mouthpiece comprising:
a U-shaped, in-mouth member configured to be worn in a user's mouth, the U-shaped, in-mouth member comprising a middle portion from which a pair of leg portions extend inwardly to respective end portions inside the user's mouth;
a front member extending from an outer surface of the U-shaped, in-mouth member and disposed outside of the user's mouth and defining an air passageway;
a pair of lateral breathing ports extending from outside the user's mouth through the front member and the U-shaped, in-mouth member and into the user's mouth; and
an anterior tongue advancement device disposed on an inner surface of the middle portion of the U-shaped, in-mouth member, the anterior tongue advancement device comprising one or more flexible members for receiving and holding the user's tongue in a forward position; and
a portable airflow accessory comprising a housing and an adapter, the housing containing a source of airflow powered by a battery, the adapter disposed on the housing for removably securing to the mouthpiece, the adapter comprising a pair of lateral members configured to be removably secured in the pair of corresponding lateral breathing ports disposed on the mouthpiece, and a center securing member configured to be removably secured in the air passageway disposed on the mouthpiece.

23. The sleep-aid system of claim 22, further comprising a nasal spreader accessory removably attached to the mouthpiece.

24. The sleep-aid system of claim 23, wherein the nasal spreader accessory comprises:
a tether member having a first end for removably attaching to the front member of the sleep-aid mouthpiece; and
a flexible transverse member fixed to a second end of the tether member, the flexible transverse member comprising a pair of nostril members adapted to exert a force in a flexed state when the respective nostril members are inserted in the user's nostrils.

25. The sleep aid system of claim 22, wherein the portable airflow accessory comprises one or more airflow ports disposed on an upper portion of the housing and in communication with the source of airflow.

26. The sleep-aid system of claim 25, wherein the one or more airflow ports are adapted to be fixed to one or more corresponding nostril cones configured for insertion in the user's nostrils while the user is wearing the sleep-aid mouthpiece and the adapter is removably secured to the sleep-aid mouthpiece.

27. The sleep-aid system of claim 26, wherein the one or more nostril cones comprise a baffled nostril cone accessory removably attached to the one or more airflow ports.

28. The sleep-aid system of claim 22, wherein the sleep-aid mouthpiece is adapted to receive an anti-drool device.

29. The sleep-aid system of claim 28, wherein the anti-drool device comprises a sterilizable elongate member having a first end configured to be received in an opening in the anterior tongue advancement device and a second end fixed to an absorbent material positionable under the user's tongue.

30. The sleep-aid system of claim 29, wherein the front member of the sleep-aid mouthpiece comprises a further opening for receiving the first end of the sterilizable elongate member of the anti-drool device.

31. A nasal spreader accessory for a sleep-aid mouthpiece, the nasal spreader accessory comprising:
a tether member having a first end for removably attaching to a front member of a sleep-aid mouthpiece; and
a flexible transverse member disposed on a second end of the tether member, the flexible transverse member comprising a pair of nostril members adapted to exert a force in a flexed state when the respective nostril members are inserted in the user's nostrils.

32. The nasal spreader accessory of claim 31, wherein the first end of the tether member comprises a retaining ball member.

33. The nasal spreader accessory of claim 31, wherein the tether member and the flexible transverse member comprise different materials.

34. The nasal spreader accessory of claim 31, wherein the pair of nostril members comprise angled flat paddles.

35. The nasal spreader accessory of claim 34, wherein each of the angled flat paddles comprise a first surface having one or more trim ridges for reducing a size of the angled flat paddles.
